(19) United States
(12) Patent Application Publication
(10) Pub. No.: US 2026/0335339 A1
(43) Pub. Date: Oct. 29, 2026

(54) COMPOSITIONS FOR DERMAL/TRANSDERMAL DELIVERY AND COSMETIC USE

(71) Applicant: YISSUM RESEARCH DEVELOPMENT COMPANY OF THE HEBREW UNIVERSITY OF JERUSALEM LTD., Jerusalem (IL)

(72) Inventors: Elka TOUITOU, Zichron Yaacov (IL); Hiba NATSHEH, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 19/110,258

(22) PCT Filed: Sep. 11, 2023
(86) PCT No.: PCT/IL2023/050976

§ 371 Date: Mar. 10, 2025

Related U.S. Application Data
(60) Provisional application No. 63/405,665, filed on Sep. 12, 2022.

(51) Int. Cl. A61K 31/00; A61K 9/00; A61K 9/06; A61K 31/351; A61K 47/02; A61K 47/10

(52) U.S. Cl. CPC A61K 31/658; A61K 9/0014; A61K 9/06; A61K 31/351; A61K 47/02; A61K 47/10

(57) ABSTRACT

The present disclosure generally relates to terpene-based vesicular delivery systems that are substantially phospho-lipid-free, and which are suitable for dermal and/or transdermal applications. The compositions provided by the invention comprise at least one active agent in a vesicular carrier, wherein the vesicular carrier comprises at least one terpene, at least one saponin, at least one C2-C4 alcohol and water, wherein said composition is substantially phospholipid-free.

### FIELD OF THE INVENTION

The present invention generally relates to compositions for delivering compounds, such as lipophilic compounds, dermally and/or transdermally.

### BACKGROUND OF THE INVENTION

The skin is the largest organ of the body and protects it from exogenous materials. The outermost layer of the skin, the stratum corneum (SC), consists of nonviable, keratinized cells lacking a nucleus and forms an effective barrier to retain water within the body, while keeping exogenous compounds out.

Plant extracts and natural products, such as terpenes, have been incorporated into skin and hair care preparations over the years, for various applications. WO 2001/095726 relates to a parasiticidal, hair-treatment formulation comprising eugenol, soapwort, water and isopropyl alcohol in a weight ratio of 0.5:0.5:30:69, respectively, having a parasiticidal action for the treatment and/or prevention of human hair infestation by parasites.

### SUMMARY OF THE INVENTION

It has been found by the inventors that there is no need of phospholipid to form a nano-vesicular delivery system; the inventors found that various combinations of terpenes and additional ingredients could generate vesicles (e.g., nano-vesicles) in aqueous/alcoholic system in the absence of phospholipids. It has also been found that these vesicular systems enhance skin penetration of various active agents, for example of cannabinoids incorporated into these compositions, and therefore these terpene-based compositions may be used as vesicular carriers or vehicles for various drugs (active agents) to and across the skin, namely, potential dermal and transdermal delivery systems. These terpene-based compositions in aqueous/alcoholic systems are also referred to herein as “terpenosomes”.

Experimental work conducted in support of the invention indicates (among others) that the ability of terpenosomes to enhance the delivery of the active molecules across the skin layers can be modulated with the aid of auxiliary additives, such as a long-chain alcohol (e.g., cetyl alcohol, which assists in delivery to the top or upper layers of the skin) and magnesium salts (such as magnesium chloride, which assists in transporting the active molecule into deeper skin layers). These and other effects attest to the adaptability of terpenosome over a wide range of dermal/transdermal applications, as further detailed below.

The terpenosomes as herein defined comprise one or more of each of terpene(s) (e.g., non-saponin terpene(s)), saponin(s), short-chain volatile alcohol(s) and water. As mentioned above, a long chain aliphatic alcohol can also be included in the composition (by alcohol we mean monohydric alcohols) or a magnesium salt. The terpenosomes may optionally further comprise polyols such as glycols. Terpenosomes constitute the major aspect of the invention. It should be noted that the terpenosomes are useful in their own right (e.g., to deliver terpenes possessing desired action dermally or transdermally), or as carriers for non-terpene active agents.

In other words, the present disclosure provides a composition comprising at least one active agent in a vesicular carrier formed by least one terpene, at least one saponin, at least one C₂-C₄ alcohol and water, wherein the composition generates a vesicular system which is substantially phospholipid-free. In various embodiments the composition as herein defined is for dermal/transdermal delivery. In some embodiments the active agent is selected from analgesic agents and/or agents for treating and/or preventing at least one skin-related disease or condition. In further specific embodiments, the active agent is a cannabinoid.

In specific embodiments the vesicular composition according to the present disclosure comprises:
from about 0.005% to about 10% by weight of at least one active agent selected from analgesics, e.g., a cannabinoid, lidocaine and ibuprofen, agents for treating and/or preventing at least one skin-related disease or condition and melatonin;
from about 0.1% to about 20% by weight of at least one terpene selected from monoterpenes and sesquiterpenes;
from about 0.1% to about 5% by weight of at least one saponin;
from about 15% to about 80% by weight of ethanol;
when present, from about 0.05% to about 5% by weight of long chain alcohol;
when present, up to 60% by weight propylene glycol and/or 1,3-propanediol;
when present, from about 0.01% to about 0.5% by weight of magnesium salt;
and
from about 5% to about 85% by weight of water.

The terpenosomes and pro-terpenosomes as herein defined are suitable for delivering agents useful for multiple conditions and disorders, inter alia, skin-related conditions and disorders. Furthermore, the vehicles described in the present disclosure are of potential use for transdermal delivery of drugs, i.e., for systemic treatment of various diseases, conditions and disorders, e.g., for transdermal delivery of analgesic agents.

Therefore by a further aspect thereof, the present disclosure provides a method for treatment and/or prevention of a disease or condition in a subject comprising administering to the skin of said subject a therapeutically effective and/or prophylactically effective amount of a composition as herein defined.

Still further, the present disclosure provides a composition as herein defined for use in a method for treatment and/or prevention of a disease or condition in a subject, said method comprising administering to the skin of said subject a therapeutically effective and/or prophylactically effective amount of said composition.

In some embodiments the condition encompassed by the present disclosure is pain and the active agent is an analgesic, e.g., at least one cannabinoid. In other embodiments the disease or condition encompassed by the present disclosure is associated with upper skin layers, such as Acne, Rosacea, skin infection, such as a microbial infection, for example a viral infection, Contact dermatitis, Atopic dermatitis, Actinic keratosis, skin inflammation, Psoriasis, Desquamation, Exfoliation, a cosmetic condition, damaged skin barrier, or dehydrated skin. In further embodiments the disease or condition is associated with deeper skin layers, such as Acne, pain, Alopecia Areata, Raynaud's Phenomenon, Vitiligo, Scleroderma, Hidradenitis suppurativa (Acne inversa), Melanoma, Squamous cell carcinoma, Basal cell carcinoma, Cellulitis, Pemphigus, Psoriasis, skin regeneration, Collagen activation, skin pigmentation or skin hyperpigmentation.

Still further the present disclosure provides a method of cosmetic treating and/or prevention of at least one skin condition in a subject, comprising applying to the skin of said subject a composition as herein defined.

The present disclosure further provides a method for preparing a composition as herein described, comprising:
a) mixing in at least one C₂-C₄ alcohol at least one terpene (e.g., non-saponin terpene) and at least one saponin thereby forming an organic phase;
b) combining the organic phase obtained in step a) with water, thereby obtaining a vesicular carrier; and
c) combining at least one active agent with the organic phase obtained in step a), the water supplied at step b) or with the vesicular carrier obtained in step b).

The term “pro-terpenosome” is used herein to indicate the organic component of terpenosome. That is, pro-terpenosomes have a similar composition to terpenosomes, but do not include water. By adding water to pro-terpenosomes, terpenosomes are formed. An active agent-added pro-terpenosome forms another aspect of the invention.

In other words, the present disclosure further provides an organic precursor, which, upon combination with water, forms a vesicular system, comprising at least one active agent selected from cannabinoids and agents for treating and/or preventing at least one skin-related disease or condition, at least one non-saponin terpene, at least one saponin, at least one C₂-C₄ alcohol, wherein said vesicular system is substantially phospholipid-free.

Still further the present disclosure provides a phospholipid-free vesicular composition comprising at least one terpene (e.g., non-saponin terpene), at least one saponin, at least one C₂-C₄ alcohol and water, wherein the composition further comprises a long chain alcohol and/or a magnesium salt, wherein the composition is devoid of non-terpene active agent.

### BRIEF DESCRIPTION OF THE DRAWINGS

Embodiments will now be described, by way of non-limiting example only, with reference to the accompanying drawings, in which:

FIG. 1: Scanning electron microscopy (SEM) micrograph showing Terpenosome vesicles of a composition described in Example 12, magnitude ×8 k, FEI Quantal 200 SEM.

FIG. 2: SEM micrograph showing Terpenosome vesicles comprising geraniol:glycyrrhizin:ethanol:double distilled water (DDW) at a weight ratio of 10:2:70:17.5, magnitude ×8 k, FEI Quantal 200 SEM.

FIG. 3: SEM micrograph showing Terpenosome vesicles comprising cannabidiol (CBD):geraniol:glycyrrhizin:ethanol:DDW at a weight ratio of 5:9.5:1.9:66.5:17.1, magnitude ×40 k, FEI Quantal 200 SEM.

FIG. 4: SEM micrograph showing Terpenosome vesicles comprising cetyl alcohol:eucalyptol:glycyrrhizin:ethanol:DDW at a weight ratio of 0.5:10:2:70:17.5, magnitude ×20 k, FEI Quantal 200 SEM.

FIG. 5: SEM micrograph showing a control composition lacking terpene, comprising cetyl alcohol:terpene:glycyrrhizin:ethanol:DDW at a weight ratio of 0.5:0:1:70:17.5, magnitude ×20 k, FEI Quantal 200 SEM.

FIG. 6: SEM micrograph showing a control composition lacking terpene, comprising CBD:glycyrrhizin:ethanol:DDW at a weight ratio of 5:1.9:66.5:17.1, magnitude ×2 k, FEI Quantal 200 SEM.

FIG. 7: SEM micrograph showing a control composition lacking terpene and glycyrrhizin, comprising CBD:ethanol:DDW at a weight ratio of 5:70:17.5, magnitude ×20 k, FEI Quantal 200 SEM.

FIG. 8: SEM micrograph showing a control composition lacking terpene and CBD, comprising glycyrrhizin:ethanol:DDW at a weight ratio of 2:70:17.5, magnitude ×2.4 k, FEI Quantal 200 SEM.

FIG. 9A-FIG. 9B: Exemplary transmission electron microscopy (TEM) micrographs of Terpenosome vesicles comprising geraniol (prepared as described in Example 128a).

FIG. 10A-FIG. 10B: Exemplary TEM micrographs of Terpenosome vesicles comprising geraniol and magnesium (prepared as described in Example 128b).

FIG. 11A-FIG. 11B: Exemplary TEM micrographs of Terpenosome vesicles comprising geraniol and CBD (prepared as described in Example 128c).

FIG. 12A-FIG. 12B: Exemplary TEM micrographs of Terpenosome vesicles comprising geraniol, magnesium and CBD (prepared as described in Example 128d).

FIG. 13: Representative confocal laser scanning (CLS) micrographs of area sections of skin treated with terpenosome composition containing FITC and limonene at the indicated skin depths, using Zeiss LSM 710 laser scanning microscopy system, Zeiss, Germany, at a stack scanning mode, 1% laser intensity, 10 m increments through the z-axis with an air plane ×10 objective lens and 488 nm excitation wavelength.

FIG. 14: Representative CLS micrographs of area sections of skin treated with terpenosome composition containing FITC, limonene and magnesium at the indicated skin depths, using Zeiss LSM 710 laser scanning microscopy system, Zeiss, Germany, at a stack scanning mode, 1% laser intensity, 10 m increments through the z-axis with an air plane ×10 objective lens and 488 nm excitation wavelength.

FIG. 15: Representative CLS micrographs of area sections of skin treated with a control non-vesicular composition containing FITC and limonene at the indicated skin depths using Zeiss LSM 710 laser scanning microscopy system, Zeiss, Germany, at a stack scanning mode, 1% laser intensity, 10 m increments through the z-axis with an air plane ×10 objective lens and 488 nm excitation wavelength.

FIG. 16: Mean fluorescence intensity (AU) measured following administration of 1. Terpenosome composition containing limonene; 2. Terpenosome composition containing limonene and magnesium; 3. Control non-vesicular composition containing the same terpene concentration Mean±SD, n 4/group. p>0.05 (considered nonsignificant) for terpenosome containing limonene vs. terpenosome containing limonene and magnesium. p<0.05 (considered significant) for terpenosome containing limonene and for terpenosome containing limonene and magnesium vs. control emulsion containing limonene by One-way ANOVA and Bonferroni's Multiple Comparison Test.

FIG. 17: Representative CLS micrographs of area sections of skin samples treated with terpenosome composition containing FITC and geraniol at the indicated skin depths.

FIG. 18: Representative CLS micrographs of area sections of skin samples treated with terpenosome composition containing geraniol and magnesium at the indicated skin depths.

FIG. 19: Representative CLS micrographs of area sections of skin samples treated with a non-vesicular control composition containing FITC and geraniol.at the indicated skin depths.

FIG. 20: Mean fluorescence intensity (AU) measured following administration of 1. Terpenosome composition containing geraniol; 2. Terpenosome composition containing geraniol and magnesium; and 3. Non-vesicular control composition containing the same terpene concentration Mean±SD, n 4/group. p>0.05 (considered nonsignificant) for terpenosome containing geraniol vs. terpenosome containing geraniol and magnesium. p<0.05 (considered significant) for terpenosome containing geraniol and for terpenosome containing geraniol and magnesium vs. control emulsion containing geraniol, by One-way ANOVA and Bonferroni's Multiple Comparison Test.

FIG. 21: Representative CLS micrographs of area sections of skin treated with terpenosome containing FITC and mixed terpenes composition at the indicated skin depths using Zeiss LSM 710 laser scanning microscopy system, Zeiss, Germany, at a stack scanning mode, 2% laser intensity, 10 m increments through the z-axis with an air-plane ×10 objective lens and 488 nm excitation wavelength.

FIG. 22: Representative CLS micrographs of area sections of skin treated with terpenosome composition containing FITC and limonene at the indicated skin depths using Zeiss LSM 710 laser scanning microscopy system, Zeiss, Germany, at a stack scanning mode, 2% laser intensity, 10 m increments through the z-axis with an air-plane ×10 objective lens and 488 nm excitation wavelength.

FIG. 23: Representative CLS micrographs of area sections of skin treated with composition containing FITC and geraniol at the indicated skin depths using Zeiss LSM 710 laser scanning microscopy system, Zeiss, Germany, at a stack scanning mode, 2% laser intensity, 10 m increments through the z-axis with an air-plane ×10 objective lens and 488 nm excitation wavelength.

FIG. 24: Mean fluorescence intensity (AU) measured following administration of 1. Terpenosome composition containing mixed terpenes (limonene, geraniol, menthol and eucalyptol); 2. Terpenosome composition containing limonene; and 3. Terpenosome containing geraniol, Mean±SEM, n 4/group. p>0.05 (considered nonsignificant) for Terpenosome containing mixed terpenes vs. Terpenosome containing limonene and vs. Terpenosome containing geraniol. p<0.05 (considered significant) Terpenosome containing limonene vs. Terpenosome containing geraniol, by One-way ANOVA and Bonferroni's Multiple Comparison Test.

FIG. 25: Mean writing counts in mice treated with 100 mg/kg CBD topically from terpenosome containing 5% limonene and 5% CBD (TERP-LIM-CBD), terpenosome containing 5% limonene, 5% CBD and magnesium (TERP-Mg-LIM-CBD), control non-terpenosome emulsion containing 5% limonene and 5% CBD (Control Emulsion-LIM-CBD), 1, 4, 8 and 12 hours prior to IP injection of acetic acid and compared to untreated control mice, mice received IP injection of acetic acid (n=7/group) (Mean±SD). p<0.001 for TERP-LIM-CBD vs. Control Emulsion-LIM-CBD, and vs Untreated. p<0.01 for TERP-Mg-LIM-CBD vs. Control Emulsion-LIM-CBD, and p<0.001 for TERP-Mg-LIM-CBD vs. Untreated. p>0.05 for Control Emulsion-LIM-CBD vs. Untreated, by One-way ANOVA and Bonferroni's Multiple Comparison Test.

FIG. 26: Mean writing counts in mice pain model treated with 100 mg/kg CBD topically from Terpenosome comprising 5% mixed terpenes and CBD (Occluded), Terpenosome comprising 5% mixed terpenes and CBD (Non-occluded), Control non-terpenosome Emulsion containing 5% mixed terpenes (Non-occluded) (n=6/group)(Mean±SD). p<0.05 for Terpenosome compositions containing mixed terpenes (occluded and non-occluded) vs. Control Emulsion (non-occluded) and untreated mice group.

FIG. 27: Representative confocal laser scanning (CLS) micrographs of area sections at the indicated skin depths of skin treated with a terpenosome composition containing cetyl alcohol, described in Example 134.

FIG. 28: Representative CLS micrographs of area sections at the indicated skin depths of skin treated with a terpenosome composition without cetyl alcohol, described in Example 134.

FIG. 29: Mean fluorescence intensity measured following application of terpenosome compositions with and without cetyl alcohol, as described in Example 134. Mean±SEM, n 3/group.

FIG. 30A-FIG. 30D: Transmission electron (TE) micrographs of terpenosome compositions prepared without cetyl alcohol (FIG. 30A-FIG. 30B) or with 0.5% cetyl alcohol (FIG. 30C-FIG. 30D) described in Example 135.

### DETAILED DESCRIPTION OF THE INVENTION

We have surprisingly found that terpenes form vesicles when combined with a saponin, ethanol and water, in the absence of phospholipids. These vesicular compositions were found to enhance the penetration into and across the skin of various compounds incorporated therein, such as cannabinoids. The present disclosure therefore provides a vesicular composition or system, for use in dermal/trans-dermal administration of active agents. In other words, the present disclosure provides a vesicular composition for enhancing the delivery or permeation of active agents to or through the skin.

Accordingly, in a first aspect thereof, the present disclosure provides a composition comprising at least one (non-terpene) active agent in a vesicular carrier, wherein the vesicular carrier comprises at least one terpene, at least one saponin, at least one C₂-C₄ alcohol and water, wherein said composition is substantially phospholipid-free.

By “substantially phospholipid-free” it is meant that the concentration of phospholipids in the composition is well below the acceptable amount used for the preparation of vesicular compositions, e.g., not more than 1.0% by weight, more preferably, up to 0.5% by weight, e.g., from about 0.0% to about 0.3% by weight, for example, between about 0.0% to about 0.1% by weight (based on the total weight of the composition). Most preferably, the compositions of the invention are completely devoid of phospholipids.

As known in the art, terpenes are lipids (hydrocarbon compounds) produced by a variety of plants, built of five-carbon isoprene (CH₂C(CH₃)CHCH₂) units. Any terpene is suitable for preparing the compositions of the present disclosure (the term “terpene”, as used herein, is meant to include non-saponin terpenes). Modified terpenes (i.e., terpene derivatives) are also contemplated. Terpenes suitable for use in the preparation of the composition of the invention include monoterpenes, sesquiterpenes and diterpenes, etc., (less than C₃₀).

Preferably, the terpenes suitable for use in preparing the compositions of the invention are monoterpenes or sesquiterpenes. Monoterpenes according to the present disclosure may be acyclic monoterpenes (i.e., linear monoterpene) or cyclic monoterpene (i.e., ring-containing monoterpenes). Preferably, the acyclic monoterpenes according to the present disclosure contain one or more oxygen atoms, for example but not limited to geraniol, citronellol and linalool. Cyclic monoterpene according to the present disclosure may be devoid of oxygen atoms (e.g., limonene) or contain one or more oxygen atoms which may be ring heteroatom(s), namely incorporated into the ring (e.g., eucalyptol) or attached to aliphatic ring carbon (e.g., menthol). The ring(s) in the cyclic monoterpene used herein are generally non-aromatic. The cyclic monoterpene according to the present disclosure may contain one or more hydroxyl groups attached to aliphatic ring carbon.

Other terpenes suitable for preparing the compositions as herein defined include, but are not limited to, monoterpenes such as Beta-Myrcene, Alpha-Pinene, Cis-Ocimene, Beta-Curcumene, Terpinolene, Camphene, Trans-Ocimene, Alpha-Terpinene, Piperitone, Eucalyptol, Linalool, Fencyl alcohol, Bete-Pinene, Citronellol, Borneol, Citronellal, D/L-Fenchone, Geraniol, D-Limonene, 3-Carene, Geranyl acetate, Cuminaldehyde, Alpha-Phellandrene, Alpha-Thujone, D/L-Menthol, Linalyl Acetate, Isopulegol, Carvone, Carvacal, Gamma Terpinene, Menthofuran, Sabinene Hydrate, Nerol, Sabinene, Thymol, Camphor, Pulegone, Bornyl acetate, Alpha-Terpinol and sesquiterpenes such as Alpha-Bisabolol, Caryophyllene oxide, M-Cymene, Isocannabispiran, Beta-Farnesene, Isoborneol, Cis-Citral, Beta-Caryophyllene, Ledene, α-Curcumene, Alpha-Humelene, Alpha-Gurjunene, Trans-Nerolidol, Beta-Cadrene, Valencene, Alpha Cedrene, Thujopsene, Alpha-Farnesene, Cis-Nerolidol, Cedrol, Guaiazulene, Farnesol, Cuparene, Isolongifolene, Guaiol or any combination thereof. Particularly suitable sesquiterpenes are Alpha-Bisabolol and/or Caryophyllene oxide.

In particular, the compositions of the present disclosure comprise any one of limonene, geraniol, menthol, eucalyptol, linalool, citronellol or a combination thereof. Mixtures consisting of two or more terpenes (e.g., >2, >3, >5, >10) could also be formulated as terpenosomes and pro-terpenosomes, for example, to deliver a mixture of terpenes possessing desired action dermally or transdermally, optionally devoid of non-terpene active agent.

The concentration of the terpenes or any modification or derivative thereof in the composition encompassed by the present disclosure is preferably in the range of from about 0.01% to about 20% by weight, e.g., from about 0.1% to about 20% by weight, from about 2.5% to about 20% by weight, for example, from 2.5% to 15%, from 2.5% to 10%, from 2.5 to 5% or from 5% to 15% by weight, based on the total weight of the composition. Terpenes as herein defined are readily available commercially or by extracting thereof from suitable starting materials as known to a person of skill in the art.

As known in the art, saponins are a subclass of terpenoids, having amphipathic nature. Any saponin is suitable for preparing the pro-terpenosome and/or terpenosome compositions of the present disclosure. By way of example, saponins suitable for use include glycyrrhizin (C₄₂H₆₂O₁₆ having a molar mass of 822.94 gr/mol), a ginsenoside, a Quillaja saponin or any combination thereof and are commercially available. In particular, the compositions of the present disclosure comprise glycyrrhizin. The concentration of the saponins in the composition encompassed by the present disclosure, e.g., for use in enhancing dermal/transdermal permeation of at least one active agent comprised in the composition is about 0.1% to about 5% by weight, preferably in the range of about 1% to about 5% by weight.

As detailed above, the vesicular composition according to the present disclosure comprises at least one C₂-C₄ alcohol (i.e., monohydric alcohol), referring to alkanols containing two, three or four carbon atoms. These short chain volatile alcohols (C₂-C₄ alcohols) to be used according to the present invention specifically include ethanol (CH₃CH₂OH), 1-propanol (CH₃CH₂CH₂OH), isopropyl alcohol (isopropanol, (CH₃)₂CHOH) and tert-butyl alcohol ((CH₃)₃COH). Preferably, the C₂-C₄ alcohol is ethanol. The concentration of the C₂-C₄ alcohol in the composition encompassed by the present disclosure is about 15% to about 80% by weight, preferably in the range of about 40% to about 80% by weight.

The inventors have found that compositions of the invention are suitable for incorporating both hydrophilic and lipophilic molecules (active agents), including cannabinoids and endocannabinoids. It has been further found that the terpenosomes compositions enhanced the delivery or permeation of cannabinoids to and across the skin.

More specifically, the terpenosome compositions of the present disclosure possess enhanced skin penetration properties compared to control, non-vesicular compositions, as evidenced by in vitro skin-penetration assays showing delivery of compounds into skin layers having a depth of at least 350 μm.

Furthermore, as exemplified below in an animal model, the terpenosome compositions of the present disclosure enhance the analgesic effect of CBD as compared to a control composition comprising CBD. The weak effect demonstrated by the control composition, containing the same concentration of terpene and CBD, emphasizes the important role of the terpenosome system in drug delivery.

Any active agent may be incorporated in the vesicular composition according to the invention, and the vesicular compositions as herein defined may be used for enhancing dermal/transdermal permeation of any active agent comprised therein, of either hydrophilic or lipophilic nature.

For example, and as exemplified herein, the composition according to the present disclosure comprises at least one cannabinoid or endocannabinoid for which ample therapeutic effects have been reported, e.g., as detailed in Table 1 below. For example, the present disclosure encompasses use of cannabinoids, e.g., cannabidiol (also known as CBD or cannabidiolum), tetrahydrocannabinol (THC), cannabinol (CBN), cannabigerol (CBG) and cannabichromene (CBC) as extracts, or synthetic cannabinoids. Furthermore, mixtures of cannabinoids may be used. Cannabinoid derivatives, e.g., of CBD are also encompassed by the present disclosure. Endocannabinoids for use in the invention include anandamide and its synthetic analogues, such as but not limited to methanandamide. In particular, the present disclosure encompasses incorporation of cannabidiol (CBD) into the compositions as herein defined and use of the resulting compositions. Generally, cannabinoid and/or endocannabinoid are commercially available. A skilled artisan is acquainted with methods of extraction and/or synthetic preparation of cannabinoid and endocannabinoid.

In Table 1 there are tabulated active compounds alongside their therapeutic effect that could benefit from incorporation into terpenosome.

TABLE 1
Active compound incorporated in terpenosomes of the invention

| Category | Active compound | Therapeutic effect |
|---|---|---|
| Cannabinoids | Cannabidiol (CBD) | Non-psychoactive, analgesic, |
| | | anti-inflammatory, antiemetic, |
| | | anxiolytic, anti-depressant, anti- |
| | | convulsive, antipsychotic, |
| | | antispasmodic, sleep aid. |
| | | For treatment of multiple |
| | | sclerosis (MS), Acne, mood |
| | | disorders, neuropathic pain, |
| | | arthritis, irritable bowel |
| | | syndrome, chemotherapy |
| | | induced nausea and vomiting |
| | | and for appetite stimulation. |
| | | Cosmetic uses include: use as |
| | | antioxidant, hydrating, anti- |
| | | sebum and anti-aging. |
| | Tetrahydrocan- | Psychoactive cannabinoid |
| | nabinol | Appetite stimulant, anti- |
| | (THC) | convulsant, anti-inflammatory, |
| | | analgesic. |
| | | For alleviation of neuropathic |
| | | pain, spasticity, overactive |
| | | bladder and for management of |
| | | chemotherapy-induced nausea |
| | | and vomiting. |
| | Cannabinol (CBN) | Mildly psychoactive |
| | | cannabinoid. Sleep aid, |
| | | analgesic, appetite stimulant. |
| | | Neuroprotective and anti- |
| | | inflammatory. |
| | Cannabichromene | Anti-depressant, anxiolytic, |
| | (CBC) | analgesic, anti-inflammatory, |
| | | and for treatment of Acne. |
| | Anandamide and | Anxiolytic, memory |
| | Methanandamide | improvement, appetite stimulant, |
| | | sleep aid, and analgesic. |
| NSAIDS | Diclofenac | Analgesic and anti- |
| | Ibuprofen | inflammatory. |
| | Ketoprofen | |
| | Piroxicam | |
| | Indomethacin | |
| Steroids | Dexamethasone | Treatment of skin inflammation, |
| | Betamethasone | redness, itchiness, swelling and |
| | Colbetasone | irritation. |
| | Fluticasone | |
| | Hydrocortisone | |
| | Mometasone | |
| | Triamcinolone | |
| Antimicrobials | Erythromycin | Anti-bacterial for treatment of |
| | | Acne. |
| | Bacitracin | Anti-bacterial for prevention of |
| | | minor skin infections. |
| | Tetracycline | Anti-bacterial for treatment of |
| | | Acne. |
| | Chloramphenicol | Anti-bacterial for prevention and |
| | | treatment of surgical wounds, |
| | | suture lines and skin grafts. |
| | Polymyxin B | Anti-bacterial used in |
| | | combination with other |
| | | ingredient to treat minor wounds |
| | | (such as cuts, scrapes, burns) |
| | | and to help prevent or treat |
| | | mild skin infections. |
| | Neomycin | Anti-bacterial used in |
| | | combination with other |
| | | ingredient to treat minor wounds |
| | | (such as cuts, scrapes, burns) |
| | | and to help prevent or treat |
| | | mild skin infections. |
| | Terbinafine | Anti-fungal used to treat fungus |
| | | infections of the scalp, body, |
| | | groin (jock itch), feet (athlete's |
| | | foot), fingernails, and toenails. |
| | Griseofulvin | Anti-fungal used to treat a |
| | | number of types of |
| | | dermatophytoses (ringworm) |
| Antivirals | Acyclovir | Antiviral used for treatment of |
| | | herpes infections. |
| | Penciclovir | Antiviral used to treat the |
| | | symptoms of herpes simplex |
| | | virus infections around the |
| | | mouth (cold sores). |
| Retinols and | Retinol | Treatment of Acne, reduction of |
| retinoids | Retinyl palmitate | wrinkles, skin regeneration and |
| | Retinoid | pore minimization. |
| | Tretinoin | |
| | Adapelene | |
| Peptides | Palmitoyl pentapeptide | Antiaging, collagen production, |
| | Acetyl tetrapeptide-9 | minimize wrinkles, enhancement |
| | Acetyl hexapeptide | of smooth and firm skin, |
| | 3, 8, and 20 | improvement of skin barrier. |
| | Tripeptide 1 | |
| | Copper peptides | |

Thus, additional exemplary active agents suitable for combining with the vesicular carrier as herein defined, thereby forming the vesicular terpenosome composition of the present disclosure, are any one of, for example, anti-inflammatory (e.g., nonsteroidal anti-inflammatory drugs (NSAIDS) and steroids), antimicrobials (e.g., antibiotics, antivirals, anti-fungal and anti-parasite agents), retinols and retinoids and peptides.

Exemplary nonsteroidal anti-inflammatory drugs (NSAIDS) for pain relief and treatment of inflammatory conditions (e.g., arthritis) is any one of diclofenac, ibuprofen (the incorporation of which into the vesicular carrier of the invention is detailed below), ketoprofen, piroxicam and indomethacin. In particular, NSAIDS suitable for being incorporated into the vesicular carrier of the invention is ibuprofen. Steroidal anti-inflammatory agents and their salts, such as dexamethasone, betamethasone, colbetasone, fluticasone, hydrocortisone, mometasone and triamcinolone are used, e.g., for treatment of skin inflammation, redness, itchiness, swelling and irritation. Suitable antimicrobials, such as but not limited to, antibiotic agents, are erythromycin, bacitracin, tetracycline, chloramphenicol, polymyxin B, neomycin, terbinafine and griseofulvin. Antivirals which may be used are for example acyclovir and penciclovir. Retinols and retinoids for treatment of Acne, reduction of wrinkles, skin regeneration and pore minimization are, among others, retinol, retinyl palmitate, retinoid, tretinoin and adapalene; and peptides used for antiaging, collagen production, for minimizing wrinkles, enhancement of smooth and firm skin, and improvement of skin barrier are for example palmitoyl pentapeptide, acetyl tetrapeptide-9, acetyl hexapeptide 3, 8, and 20, tripeptide 1 and copper peptides. Further suitable active agents are, inter alia, cosmetic skin, body and/or hair care agents, such as antiaging agents, vitamins and derivatives thereof (e.g., D, E, A, C, vitamin A derivatives, retinol, retinyl palmitate, retinoid, tretinoin, panthenol (vitamin B5), niacinamide (a derivative of Vitamin B3)), for example at least one of vitamin C, vitamin D and vitamin E, the incorporation thereof into the vesicular carrier of the invention is detailed in the Examples section below, sulphur, resorcinol, adapalene, alpha lipoic acid, salicylic acid, caffeine, glycolic acid, mandelic acid, azelaic acid, squalane, alpha arbutin, hyaluronic acid, collagen peptides and synthetic peptides such as palmitoyl peptides, acetyl peptide, and copper peptides. Anti-Acne and anti-seborrhea agents are also contemplated. Some examples thereof are listed in Table 1 above.

By non-limiting specific examples, the vesicular terpenosome composition as herein defined comprises at least one agent for treating and/or preventing at least one skin-related disease or condition, such as but not limited to an anti-Acne agent (namely, an agent known to be effective against Acne, e.g., salicylic acid, tretinoin, resorcinol, retinol, adapalene, niacinamide and azelaic acid), an anti-aging agent (namely, an agent used to prevent or lessen the effects of aging, e.g., vitamin C, hyaluronic acid, retinol, alpha arbutin, alpha lipoic acid and caffeine), a skin exfoliating agent (namely, an agent involved in the removal of the old, dead skin cells from the skin's surface, e.g., salicylic acid, mandelic acid, lactic acid and glycolic acid) and an anti-wrinkles agent (namely, an agent used to promote repair of sun-damaged skin and reduce fine lines and wrinkles, e.g., niacinamide, retinol, copper peptides and hyaluronic acid) or any combination(s) thereof.

The vesicular terpenosome compositions as herein defined may comprise (or the vesicular carrier may be combined with) said at least one anti-Acne agents, anti-aging agents, skin exfoliating agents and anti-wrinkles agents (or any combination thereof) as the active agent or in addition to another active agent, such as the at least one cannabinoid or endocannabinoid.

It should be noted that the active agent can be a chemically defined synthetic molecule, a naturally derived or synthetic peptide, a protein, a polysaccharide or a nucleic acid. The active agent may also be referred to as active compound, drug, drug substance, medicinal substance, therapeutic (active) agent, cosmetic active agent and the like. The active agents can be dermally/trans-dermally delivered by means of the above compositions (vesicular carriers) as such or in admixture with additional excipients or solvents, thereby forming a suitable topical dosage form (e.g., a cream, an ointment, a gel, etc.).

When present in the compositions of the invention, the at least one active agent (e.g., the cannabinoid) is at a concentration of about 0.005% to about 10% by weight, e.g., about 0.01% to about 5%.

As detailed above, the compositions of the invention optionally further comprise additional components. Preferably, the additional component is at least one long chain aliphatic alcohol. Long chain aliphatic alcohols suitable for preparing the composition of the present invention include alcohols having a chain of 13 to 21 carbon atoms, such as but not limited to cetyl alcohol (a C-16 fatty alcohol having the formula CH₃(CH₂)₁₅OH), stearyl alcohol (C-18), cetosearyl alcohol (a mixture of fatty alcohols, consisting predominantly of cetyl (C-16) and stearyl alcohols (C-18)), or any combination thereof.

In particular, the composition of the present invention includes a long chain aliphatic alcohol such as cetyl alcohol (CH₃(CH₂)₁₅OH) which was shown by the inventors to modulate the delivery of the molecules incorporated into the terpenosome compositions of the invention. The presence of a long chain alcohol such as cetyl alcohol in the vesicular carrier favors delivery to upper skin layers, as shown below, and therefore a long chain alcohol such as cetyl alcohol is suitable as an auxiliary agent for preparing terpenosome delivering active agents for treatment and/or prevention of a disease or condition associated with upper skin layers. The concentration of the long chain aliphatic alcohols in the composition encompassed by the present disclosure is from about 0.05% to about 5% by weight, e.g., from 0.1% to about 5% by weight, e.g., from 0.1% to about 2%, from 0.1% to about 1%, from 0.2% to about 0.5% by weight.

At least one polyol (i.e., an organic compound containing multiple hydroxyl groups), such as but not limited to glycol (i.e., an alcohol having two hydroxyl groups) may also be added to the compositions as herein defined. By way of example, polyols suitable for preparing the compositions as herein defined are propylene glycol (CH₃CH(OH)CH₂OH), 1,3-propandiol (CH₂(CH₂OH)₂), diethylene glycol monoethyl ether (Transcutol®), glycerin (HOCH₂CH(OH)CH₂OH) or any combination thereof, all of which are commercially available. Preferably, the at least one polyol added to the compositions as herein defined is propylene glycol and/or 1,3-propandiol. The concentration of the polyols in the composition encompassed by the present disclosure is up to 60% by weight, such as from about 1% to about 40% by weight or from about 5% to about 30% by weight.

It was pointed out above that the presence of magnesium salts such as magnesium chloride as an auxiliary agent in terpenosome can facilitate delivery of active agents into deeper layers of the skin. Therefore, magnesium salts such as magnesium chloride are suitable as an auxiliary agent for preparing terpenosome delivering active agents for treatment and/or prevention of a disease or condition associated with deeper skin layers. The concentration of the magnesium salt is in the range of 0%-0.5%, such as 0.01% to about 0.5% by weight, in the range of 0.05%-0.5%, or 0.1%-0.2%.

The terpenosome compositions of the present disclosure may be prepared by mixing together the various ingredients, namely water, terpene(s), saponin(s) and short chain alcohols and any additional components if present, under conditions that allow the formation of vesicles. More specifically, the compositions of the present disclosure may be prepared by dissolving the terpene(s) and saponin(s) in the short chain alcohol(s), simultaneously or consecutively, preferably under stirring (e.g., overhead stirrer, Heidolph RZR 2000 Digital, Heidolph Instruments, Germany) at a speed of between about 200 to 800 revolutions per minute (RPM) typically at room temperature or at an elevated temperature (preferably not higher than 50 °C), to form the pro-terpenosome composition, with the subsequent addition of water. Further, homogenization or size reducing procedures can be used. When present, long chain aliphatic alcohols and/or polyols are dissolved in the organic component at this stage, through mixing, until homogenous solution is obtained and then the organic component is combined with the aqueous component. When present, the non-terpene active agent, such as a cannabinoid is usually added to the organic phase (pro-terpenosome) and mixed well with the other components before the addition of water. But the order of steps could be changed, i.e., a water-soluble active agent may be incorporated into the vesicular composition through the aqueous phase.

Therefore, the present disclosure further provides a method for preparing a composition as described above, comprising:
a) mixing in at least one C₂-C₄ alcohol at least one terpene and at least one saponin thereby forming an organic phase;
b) combining the mixture obtained in step a) with water, thereby obtaining a vesicular carrier; and
c) combining at least one active agent with the mixture (organic phase) obtained in step a), (i.e., a lipophilic active agent); with the water supplied at step b) (i.e., an hydrophilic active agent), or with the vesicular carrier obtained in step b).

By way of example, the vesicular composition as herein defined comprises:
from about 0.005% to about 10% by weight of at least one active agent selected from cannabinoids, and agents for treating and/or preventing at least one skin-related disease or condition;
from about 0.01% to about 20% by weight of at least one terpene, e.g., from about 0.1% to about 20% by weight of at least one terpene, e.g., monoterpene; from about 0.1% to about 5% by weight of at least one saponin;
from about 15% to about 80% by weight of ethanol;
when present, from about 0.05% to about 5% by weight of long chain alcohol, e.g., C₁₃-C₂₁ alcohol;
when present, up to 60% by weight propylene glycol and/or 1,3-propanediol; when present, from about 0.01% to about 0.5% magnesium salt;
and
from about 5% to about 85% by weight of water.

Further (non-limiting) exemplary compositions that may be prepared in accordance with the invention are listed in Table 2 below. It is to be understood that the amount of water in the compositions will be dependent on the amounts of the other components.

TABLE 2
Exemplary compositions

| | | | Exemplary |
|---|---|---|---|
| | | Range | range |
| Ingredient | Exemplary ingredients | (% w/w) | (% w/w) |
| Terpene | limonene, geraniol, | 0.01%-20% | 0.1%-12.5% |
| | menthol, eucalyptol, | | 2.5%-12.5% |
| | linalool, | | 0.1%-10% |
| | citronellol | | 0.1%-5% |
| Saponin | glycyrrhizin | 0.1%-5% | 0.2%-5% |
| | | | 0.4%-3% |
| | | | 1%-3% |
| Short chain | ethanol, isopropanol | 15%-80% | 15%-74% |
| volatile | | | 25%-65% |
| alcohol | | | |
| Long chain | Cetyl alcohol | 0.1%-1% | 0.2%-0.75% |
| aliphatic | | | 0.2%-0.5% |
| alcohol | | | |
| Salt | MgCl₂ | 0%-0.2% | 0.05%-0.2% |
| | | | 0.1%-0.2% |
| Polyol | 1,3-propanediol, | 0%-50% | 0%-60% |
| | propylene glycol | | 5%-30% |
| Active | CBD, THC, CBN, CBG, | 0%-10% | 0.1%-7.5% |
| agents | salicylic acid, mandelic | | 0.1%-5% |
| | acid, lactic acid, glycolic | | 0.5%-5% |
| | acid, tretinoin, resorcinol, | | |
| | retinol, adapalene, | | |
| | niaciamise, azelaic acid, | | |
| | vitamin C, hyaluronic acid, | | |
| | alpha arbutin 2%, alpha | | |
| | lipoic acid, caffeine, | | |
| | copper peptides | | |
| water | DDW | 5%-85% | 18%-85% |
| | | | 20%-70% |

As mention above, the combination of terpene(s), e.g., non-saponin terpene(s), saponin(s), short-chain alcohols and water in the presence or in the absence of additional agents, at the concentrations specified above, allows the formation of non-irritant compositions with vesicles present therein, whose size ranging between about 50 nm to few microns in diameter, and more specifically, up to 5 μm in diameter, e.g., up to 1 μm in diameter, as microscopically-characterized in the Examples below. For example, vesicles are shown in FIG. 1, which is a scanning electronic micrograph of a composition comprising geraniol, glycyrrhizin, ethanol and water at 10:2:70:18, respectively, by weight of the total weight of the composition (according to Example 12). It may be seen that in this specific system, the vesicular structures are of dimensions of less than one μm in diameter. The vesicular systems were visualized by transmission electron microscopy (TEM) using a Philips TECHNAI CM 120 (Eindhoven, Netherland) at 120 kV and by scanning electron microscopy (SEM) using FEI Quantal 200 SEM at acceleration voltages of 5 or 10 kV.

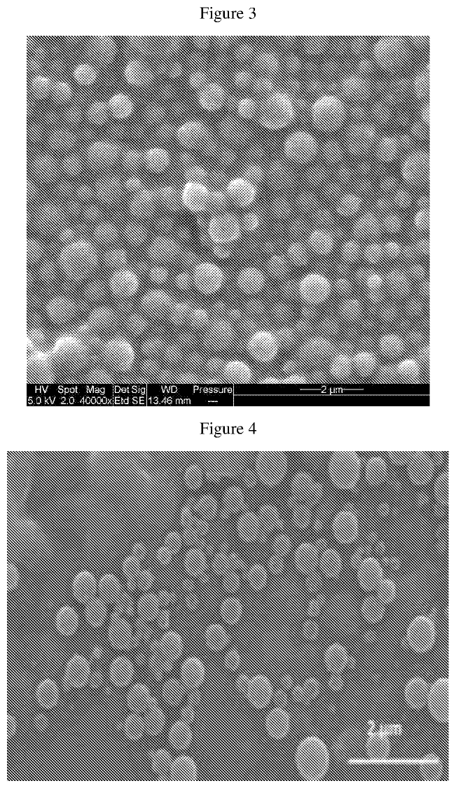

A scanning electron micrograph of a composition containing CBD is provided in FIG. 3, which represents a composition comprising CBD: geraniol:glycyrrhizin:ethanol:water at concentrations of 5:9.5:1.9:66.5:17.1% w/w, respectively. As shown, for example, in FIG. 3, it has been found that addition of a lipophilic molecules, e.g. cannabidiol (CBD) to the composition affects the vesicle size and reduces their dimensions, thereby facilitating the delivery to the skin. Hence, the composition of the invention contains nano-sized vesicles (with diameter of less than 1 μM, e.g., in the range of 100-400 nm).

The vesicular compositions of the invention can be used for pharmaceutical, cosmetic, medical, veterinary, diagnostic and research applications. As detailed above, the dermal/transdermal delivery may be intended either for local purposes (namely to skin layers, also referred to herein as “upper” (i.e., external, outer) and “deeper” (i.e., internal, viable epidermis and/or dermis) skin layers) or for systemic administration requiring transdermal delivery of the active agent. For example, the compositions may be used for transdermal delivery of pharmaceutically active agents for obtaining a systemic effect, such as by melatonin, the incorporation thereof into a composition of the invention is described below. By a further example, the compositions may be cosmetically used as skin exfoliating formulations (e.g., for removing dead skin cells from the surface of the skin), for skin cleansing or for antiaging or anti-wrinkle applications.

By the term “topical” it is meant any application to a body surface, including the skin. In particular, the present invention encompasses application to the skin of the subject, such that the agent crosses the external surface of the skin and enters deeper (i.e., viable epidermis and/or dermis) tissue. Further to the Examples below, showing permeation enhancement of the active agent by the compositions of the present invention, it should be understood that the compositions as herein defined can act as dermal/transdermal delivery vehicle(s) (vesicular carriers) of active agent(s), enabling absorption/permeation of active agents incorporated therein to upper skin layers as well as to deep skin layers, to act locally or systemically. Therefore, by topical administration of the compositions as herein defined, the at least one active agent incorporated therein has a beneficial therapeutic, prophylactic and/or cosmetic effect both locally, namely on the skin layers (i.e., on outer or inner skin layers) and systemically, upon trans-dermal permeation of the agent, reaching the systemic circulation. Thus, the composition as herein defined is for dermal/transdermal delivery.

The term subject as herein defined encompasses mammals, including humans, pet animals, laboratory animals, farm animals and wild animals.

The present invention further provides a composition as herein defined for use in a method for dermal absorption and/or transdermal permeation of at least one active agent in a subject, said method comprising topically administering to said subject the composition as herein defined.

Furthermore, the present invention provides a method for treatment and/or prevention of a disease or condition in a subject comprising topically administering (e.g., to the skin) to said subject a therapeutically effective and/or prophylactically effective amount of a composition as herein defined.

The terms “treat, treating, treatment” as used herein mean ameliorating one or more clinical indicia of disease/disorder in a subject. Providing a “preventive” or “prophylactic” treatment is acting in a protective manner, to defend against or prevent something, especially a condition or disease, at least partially.

By way of example, by the terms “disease or condition” it is referred to any disease, disorder or conditions, which may be systemic and/or localized to the skin. By way of non-limiting examples, the compositions and methods of the present disclosure are applicable to treating or preventing inflammation (e.g., skin inflammatory conditions), anxiety, infectious diseases, nausea, seizures, and various skin conditions, such as Acne, wounds, rosacea and seborrhea, dry skin among others. Exemplary active agents listed in Table 3 below are indicated according to their therapeutic effect.

TABLE 3
Therapeutic effects of active molecules incorporated in terpenosomes

| Therapeutic activity | Active compounds |
|---|---|
| Analgesia | CBD, THC, CBN, diclofenac, ibuprofen, ketoprofen, |
| | piroxica, indomethacin, lidocaine. |
| Anti- | CBD, THC, CBC, diclofenac, ibuprofen, ketoprofen, |
| inflammatory | piroxicam, indomethacin, dexamethasone, betamethasone, |
| | colbetasone, fluticasone, hydrocortisone, mometasone, |
| | triamcinolone. |
| Anxiolytic | CBD, THC, CBC, anandamide and methanandamide. |
| Anti-bacterial | Erythromycin, bacitracin, tetracycline, chloramphenicol, |
| | silver sulfadiazine, polymyxin b, neomycin |
| Anti-fungal | Terbinafine, griseofulvin. |
| Antiviral | Acyclovir, penciclovir |
| Anti-emetic | CBD, THC. |
| Anti-convulsant | CBD. |
| Anti-Acne | CBD, clindamycin, tetracycline, salicylic acid, retinol, |
| | adapelene, tretinoin, glycolic acid, azelaic acid. |
| Wound healing | CBD, hyaluronic acid, chloramphenicol, erythromycin, |
| | silver sulfadiazine. |
| Skin antiaging | CBD, retinols and retinoids, hyaluronic acid, vitamin E, |
| | vitamin C, Vitamin D, peptides, AHA, BHA. |
| Skin whitening | Retinols and retinoids. |
| Skin exfoliation | Retinols and retinoids. |
| and regeneration | |
| Minimization of skin | Peptides, retinols, retinoids, niacinamide. |
| pores and texture | |
| irregularities | |
| Treatment of skin | 5-fluorouracil (5-FU), Imiquimod, Interferon, Vismodegib, |
| cancer | Cemiplimab-rwlc. |

In particular, diseases or conditions associated with the skin are encompassed by the present disclosure, for example but not limited to Acne, an inflammatory disease of the skin, a disease resulting from at least one microbiological organism, or a condition associated with skin aging or exposure to sunlight. Preferably, the active agent in said compositions is selected from anti-Acne agents, anti-aging agents, skin exfoliating agents and anti-wrinkles agents, for example as defined herein above.

As exemplified below in an animal model, the terpenosome compositions of the present disclosure enhance the analgesic effect of CBD. Therefore, in some embodiments, the method for treatment and/or prevention of a disease or condition in a subject is wherein said condition is pain. In other words, the present disclosure further provides a method of analgesia in a subject comprising topically administering to said subject a composition as herein defined, wherein the active agent is an analgesic.

As known in the art, by the term “analgesia” it is meant relief, at least in part, from pain and by the term “analgesic” it is referred to an agent capable of exerting at least a partial pain relief effect.

Exemplary analgesics include, but are not limited to CBD, THC, CBN, diclofenac, ibuprofen, ketoprofen, piroxicam, indomethacin, and lidocaine. Preferably, the analgesic is at least one of a cannabinoid, ibuprofen, and lidocaine, in particular at least one cannabinoid (e.g., CBD) or a mixture thereof.

Still further the present disclosure provides a composition as herein defined, for use in a method of analgesia in a subject, said method comprising topically administering to said subject the composition as herein defined, wherein the active agent is an analgesic.

In particular embodiments, the present disclosure provides a method for treatment and/or prevention of a disease or condition associated with upper skin layers. As shown by the inventors and as detailed above, including long-chain fatty alcohol in the composition (e.g., cetyl alcohol) favors delivery of the active agent into upper skin layers and therefore compositions prepared as described herein which include cetyl alcohol are particularly useful for delivery of active agent to upper skin layers.

By way of example, the disease or condition associated with upper skin layers is at least one of Acne, Rosacea, skin infection, such as a microbial infection, for example a viral infection, Contact dermatitis, Atopic dermatitis, Actinic keratosis, skin inflammation, Psoriasis, Desquamation, Exfoliation, a cosmetic condition, damaged skin barrier, or dehydrated skin.

Furthermore, the present disclosure provides a method for treatment and/or prevention of a disease or condition wherein said disease or condition is associated with deeper skin layers. As shown by the inventors, including magnesium salts (such as magnesium chloride) in the compositions favors delivery of the active agent into deeper skin layers and therefore compositions prepared as described herein which include magnesium salts are particularly useful for delivery of active agent to deeper skin layers.

By way of example, the disease or condition associated with deeper skin layers is at least one of Acne, pain, Alopecia Areata, Raynaud's Phenomenon, Vitiligo, Scleroderma, Hidradenitis suppurativa (Acne inversa), Melanoma, Squamous cell carcinoma, Basal cell carcinoma, Cellulitis, Pemphigus, Psoriasis, skin regeneration, Collagen activation, skin pigmentation or skin hyperpigmentation.

The present disclosure further provides a method of cosmetic treating and/or prevention of at least one skin condition in a subject, comprising applying to the skin of said subject a composition according to the present invention. The present disclosure further provides a composition according to the present invention for use in a method of cosmetic treating and/or prevention of at least one skin condition in a subject, said method comprising applying to the skin of said subject the composition of the invention.

The inventors have also found that compositions as described above, but lacking water (also referred to herein as “pro-terpenosomes”) generate terpenosomes when water is added thereto, namely in the presence of water. The pro-terpenosome has a similar composition to terpenosome, but without water.

Thus, the present disclosure further provides an organic precursor, which, upon combination with water, forms a vesicular system, comprising at least one active agent selected from cannabinoids and agents for treating and/or preventing at least one skin-related disease or condition, at least one terpene, at least one saponin, at least one C₂-C₄ alcohol, wherein said vesicular system is substantially phospholipid-free.

By “effective” amount of an active agent or a composition is meant a sufficient amount of an active agent to provide the local or systemic effect, where the amount required to provide the effect may be determined by a skilled person.

The vesicular systems/terpenosome compositions as herein defined enhance dermal/transdermal permeation of at least one active agent comprised therein and are therefore suitable as such, namely as vehicles/carriers (or vesicular carrier) for delivery of active agent(s). The composition as herein defined may be incorporated into any dosage form suitable for topical (skin) application, including but not limited to, creams, ointments, pastes or emulsions (oil in water or water in oil), gels, lotions or foams. Preferably, the composition is formulated as a lotion, a gel, an ointment, a cream, a paste and a foam.

Preparation of such dosage forms are well known to a skilled artisan, for example, terpenosome-based gels can be obtained by adding a gelling agent (Carbopol, cellulose derivatives) or by incorporating the terpenosome compositions into a gel formulation.

Terpenosome compositions can further be used as permeation enhancers for delivery of molecules into the skin and membranes, in perfumes, eau de cologne and in mouthwashes. The terpenosome and pro-terpenosome compositions as herein defined, as well as any dosage form comprising thereof, may further include excipients, e.g., pharmaceutically-or cosmetically-acceptable excipients, well known to those versed in the art, depending on the nature of the composition/dosage form and the intended use thereof, such as salts, surfactants, preservatives, thickening agents, co-solvents, adhesives, antioxidants, buffers, viscosity and absorption enhancing agents and agents capable of adjusting the pH and osmolarity of the formulation. In particular, terpenosome and pro-terpenosome compositions as herein defined, as well as any dosage form comprising thereof, may further include salts, e.g., magnesium chloride (MgCl₂), which was shown by the inventors to enhance skin penetration of agents incorporated into the compositions in e.g., Examples 129 and 130 below.

Suitable surfactants that can be used in accordance with the present invention include ionic, nonionic or amphoteric surface active agents. More specifically, hydrophilic surfactants (e.g., Tweens, Tween 80, Myrj, Brijs, Labrasol etc.) or lipophilic surfactants (e.g., Span 20, Span 60, Span 80, Span 40, Span 65 Arlacel 83, etc.) may be suitably used, preferably at a concentration in the range of 0-25% by weight.

The compositions as herein defined may further comprise preservatives. Any preservative known to a person of skill in the art may be used. Suitable exemplary preservatives that can be used with the composition of the invention include preservatives acceptable for topical administration, for example, phenoxy ethanol, phenyl ethanol, benzyl alcohol, benzoates, benzyl benzalkonium salts, cetrimide, potassium sorbate, sorbic acid, ethyl hexyl glycerin, to name but a few.

One or more antioxidants can be added to the composition of the present disclosure, for example, at a concentration of from about 0.05% to about 1.5% by weight, based on the total weight of the composition. Suitable antioxidants include, but are not limited to, tocopherols and tocopheryl derivatives (vitamin E), 3,5-Di-tert-4-butylhydroxytoluene (BHT), butylated hydroxyaniline (BHA), lycopene, ascorbyl palmitate and the like. Mixtures of antioxidants may be used as well.

Regarding buffers, the dermal/transdermal delivery systems may include a buffer for maintaining the formulation at a pH of about 6.0-7.0. The particular buffer, of course, can vary depending upon the particular delivery system used, as well as the specific active molecule selected. Buffers that are suitable for use in the present invention include, for example, acetate, citrate, prolamine, carbonate and phosphate buffers and combinations thereof. The pharmaceutical formulations of the present invention may further include a pH adjusting agent.

Regarding thickening agents, the viscosity of the formulations of the present invention can be maintained at a desired level using a pharmaceutically acceptable thickening agent. Thickening agents that can be added to the compositions of the present invention include for example, methyl cellulose, xanthan gum, tragacanth, adhesives, guar gum, carboxymethyl cellulose, hydroxypropyl cellulose, carbomer, polyvinyl alcohol, alginates, acacia, chitosans, mucoadhesive polymer-systems like poly (acrylates), cellulose derivatives, hyaluronic acid and/or hyaluronic acid derivatives, chitin, collagen, pectin, starch, poly (ethylene glycol), sulfated polysaccharides, carrageenan, Na-alginate, gelatine, pectin and combinations thereof. The desired concentration of the thickening agent will depend upon the agent selected and the viscosity desired.

The terpenosome compositions of the present disclosure may further include emollients. Emollients suitable for inclusion in the present terpenosome compositions include, but are not limited to, esters (e.g., isopropyl palmitate, isopropyl myristate), ethers (e.g., decapryl ether and peg-7 glyceryl cocoate), triglycerides (e.g., capric and caprylic triglycerides), fatty alcohols (e.g., cetyl alcohol and oleyl alcohol), fatty acids (e.g., oliec acid and linoleic acid), hydrocarbons (e.g., liquid paraffin), vegetable butters (e.g., shea butter), vegetable oils (e.g., hemp seed oil, sesame oil, grape seed oil and jojoba oil), estrols (e.g., lanolin) and silicones (e.g. dimethicone and dimethiconol).

As mentioned above, the terpenosomes are useful in their own right (i.e., without an additional active agent), namely, to deliver terpenes possessing desired action, dermally (e.g., by inclusion of long chain alcohol in terpenosome) or transdermally (by inclusion of magnesium salt in terpenosome).

Suitable terpenes or terpene combinations having various therapeutic activities (exemplary therapeutic activities are shown in brackets) include, but not limited to, monoterpenes such as Beta-Myrcene (analgesic), Alpha-Pinene (relaxing), Cis-Ocimene (anti-fungal), Beta-Curcumene (anti-inflammatory, anti-melanogenic, anti-oxidant), Terpinolene (sedative), Camphene (reduces hyperlipidemia), Trans-Ocimene (anti-microbial), Alpha-Terpinene (antioxidant, skin repair), Piperitone (anti-fungal, antibacterial), Eucalyptol (anti-inflammatory, anti-tumor, antioxidant, anti-anxiety), Linalool (anti-anxiety, analgesic, antidepressant, sedative, sedative, anti-convulsant), Fencyl alcohol, Bete-Pinene (anti-depressant, anti-bacterial), Citronellol (antioxidant, anti-inflammatory, analgesic, hypotensive), Borneol (analgesic, anti-anxiety, anti-thrombotic), Citronellal (analgesic, sedative, mosquito repellant), D/L-Fenchone (analgesic, wound healing), Geraniol (antioxidant, antifungal), D-Limonene (analgesic, antidepressant, anti-inflammatory, antioxidant, anti-cancer), 3-Carene (anti-inflammatory, anti-fungal), Geranyl acetate (antifungal and anti-inflammatory), Cuminaldehyde (anti-cancer), Alpha-Phellandrene (anti-oxidant, anti-inflammatory, wound healing), Alpha-Thujone (anti-inflammatory, anti-fungal), D/L-Menthol (anesthetic, antidepressant), Linalyl Acetate (anti-inflammatory, anti-anxiety), Isopulegol (anti-inflammatory, enhancement of skin permeation), Carvone (analgesic, anti-convulsant, sedative, relaxing, anti-cancer), Carvacal (vasorelaxant, anti-cancer), Gamma Terpinene (antifungal, antioxidant), Menthofuran, Sabinene Hydrate (anti-cancer), Nerol (anti-fungal), Sabinene (antioxidant and anti-microbial, anti-cancer), Thymol (anti-inflammatory, wound healing, antioxidant, anti-microbial), Camphor (anti-inflammatory, increase blood flow), Pulegone, Bornyl acetate (anti-inflammatory, analgesic, relaxing), Alpha-Terpinol (local anesthetic, anti-inflammatory, anti-cancer, anti-hypertensive) and sesquiterpenes such as M-Cymene (anti-inflammatory), Isocannabispiran, Beta-Farnesene, Isoborneol, Cis-Citral (anti-tumor), Beta-Caryophyllene (anti-cancer, anti-anxiety), Ledene, α-Curcumene, Alpha-Bisabolol (anti-cancer, anti-inflammatory, sedative, analgesic, antioxidant), Alpha-Humelene (anti-inflammatory, anti-tumor, angiogenesis action), Alpha-Gurjunene (anti-inflammatory, anti-nociceptive), Trans-Nerolidol (anti-cancer, anti-fungal), Caryophyllene oxide (analgesic & anti-inflammatory, anti-microbial, anti-cancer), Beta-Cadrene (anti-inflammatory, antiseptic, and diuretic), Valencene (anti-inflammatory, skin protectant and anti-allergic), Alpha Cedrene, Thujopsene, Alpha-Farnesene, Cis-Nerolidol (Inhibition of tumors, anti-fungal, and antibacterial), Cedrol (anxiolytic), Guaiazulene (analgesic, anti-inflammatory), Farnesol (anti-inflammatory, antibacterial, and antispasmodic, anti-biofilm activity), Cuparene (anti-inflammatory, anti-bacterial), Isolongifolene and Guaiol (regulates apoptosis).

Some exemplary therapeutic effects of terpenes suitable for incorporation into the terpenosome compositions as herein defined are also listed in Table 4 below.

TABLE 4
Therapeutic effects of terpenes incorporated in Terpenosomes

| Therapeutic activity | Terpenes |
|---|---|
| Analgesic | Beta-Myrcene, Linalool, Citronellol, Borneol, Citronellal, |
| | D/L-Fenchone, D- Limonene, 3- Carene, Geranyl acetate, |
| | Carvone, Bornyl acetate, Alpha- Bisabolol, Alpha- |
| | Gurjunene, Caryophyllene oxide, Guaiazulene |
| Anti-inflammatory | Beta-Curcumene, Eucalyptol, Citronellol, D- Limonene, |
| | Alpha-Phellandrene, Alpha-Phellandrene, Linalyl Acetate, |
| | Isopulegol, Thymol, Camphor, Bornyl acetate, Alpha- |
| | Terpinol, M-Cymene, Alpha- Bisabolol, Alpha-Humelene, |
| | Alpha-Gurjunene, Caryophyllene oxide, Beta-Cadrene, |
| | Valencene, Guaiazulene, Farnesol, Cuparene |
| Antioxidant | Beta-Curcumene, Terpinene, Eucalyptol, Citronellol, |
| | Geraniol, D- Limonene, Alpha-Phellandrene, Gamma |
| | Terpinene, Sabinene, Thymol, Alpha- Bisabolol |
| Skin repair | Terpinene, D/L-Fenchone, Alpha-Phellandrene, Thymol, |
| and wound healing | |
| Anti-anxiety | Eucalyptol, Linalool, Borneol, Linalyl Acetate, Beta- |
| | Caryophyllene, Cedrol, |
| Anti-depressant | Linalool, Bete-Pinene, D- Limonene, D/L-Menthol, |
| Relaxing | Alpha-Pinene, Carvone, Bornyl acetate, |
| Sedative | Terpinolene, Linalool, Citronellal, Carvone, Alpha- |
| | Bisabolol, |
| Anti-melanogenic | Beta-Curcumene |
| Anti-cancer | Eucalyptol, D- Limonene, Cuminaldehyde, Carvone, |
| | Carvacal, Sabinene Hydrate, Sabinene, Alpha-Terpinol,, |
| | Beta-Caryophyllene, Alpha- Bisabolol, Alpha-Humelene, |
| | Trans-Nerolidol, Caryophyllene oxide, Cis-Nerolidol, |
| Anti-convulsant | Linalool |
| Anti-allergic | Valencene |
| Anti-hyperlipidemic | Camphene |
| Anti-hypertensive | Citronellol, Alpha-Terpinol, |
| Therapeutic activity | Terpenes |
| Local anesthetic | D/L-Menthol, Alpha-Terpinol, |
| Anti-thrombotic | Borneol |
| Anti-Fungal | Piperitone, Geraniol, 3- Carene, Geranyl acetate, Alpha- |
| | Phellandrene, Gamma Terpinene, Nerol, Trans-Nerolidol, |
| | Cis-Nerolidol |
| Anti-microbial | Piperitone, Trans-Ocimene, Bete-Pinene, Sabinene, Thymol, |
| | Caryophyllene oxide, Beta-Cadrene, Cis-Nerolidol, Farnesol, |
| | Cuparene |
| Apoptosis regulation | Guaiol |

Another aspect of the invention is a vesicular composition for dermal/transdermal delivery comprising at least one terpene, at least one saponin, at least one C₂-C₄ alcohol, at least one long chain alcohol and/or physiologically acceptable magnesium salt, and water, wherein said vesicular composition is substantially phospholipid-free and is devoid of non-terpene active agent.

### EXAMPLES

### Abbreviations

Example—Ex.
Ethanol (absolute)—EtOH
Cannabidiol—CBD
Tetrahydrocannabinol—THC
Cannabigerol—CBG
Cannabinol—CBN
Double distilled water—DDW
Polydispersity index—PDI
Scanning electron microscopy—SEM
Transmission electron microscopy—TEM

### Materials

Terpenes (e.g, Limonene, Geraniol, Menthol and Eucalyptol, Linalool, Citronellol) were from Sigma Aldrich, Israel. Glycyrrhizin was from TCI, Japan. Cannabidiol (CBD) was from Pureforme, USA, Fluorescein isothiocyanate (FITC) and Vitamin D were from Sigma Aldrich, Israel, Vitamin C, and Vitamin E (Tocopherol acetate) were from Tamar, Israel, Lidocaine base was from Trima, Israel, Melatonin was from Tamar, Israel, Ibuprofen was from Sigma Aldrich, Israel. Cetyl alcohol was from Holland Moran, Israel.

### Methods

### Preparation of Terpene-Based Vesicles (Also Termed Herein “Terpenosomes”)

The terpene-based vesicles referred to herein were generally prepared by dissolving in alcohol (C₂-C₄ alcohol, e.g., absolute ethanol, 70% ethanol, isopropanol or a mixture thereof) the terpene(s) (e.g., limonene, geraniol, menthol, etc.) and glycyrrhizin, simultaneously or consecutively. When present, long-chain (fatty) alcohols (e.g., ceyl alcohol) and polyols (e.g., propylene glycol and/or 1,3-propanediol) as well as lipophilic active agents (e.g., cannabinoids, Vitamin D, Vitamin E, Lidocaine, Melatonin, Ibuprofen) were dissolved in the composition at this stage through mixing, using an overhead stirrer (Hei Torque 200, Heidolph overhead stirrer, Heidolph, Germany) at a speed ranging between about 200 to 800 revolutions per minute (RPM) until homogenous solution was obtained. Finally, double-distilled water (DDW) was gradually added through mixing. Water-soluble active agents (e.g., Vitamin C) or non-active agents (e.g., MgCl₂, polymers such as hydroxypropylmethyl cellulose, etc.) were dissolved in water prior to addition thereof to the organic phase. Specific quantities and ratios of all added agents are detailed below.

### In Vitro Permeation Study Using Static Franz Diffusion Cell System

Skin permeation was tested in vitro in Franz static diffusion cells (PeremeGear, USA) as previously described (Allon, I. and Touitou, E., 2016. Scrolls: novel microparticulate systems for enhanced delivery to/across the skin. Drug delivery and translational research, 6(1), pp. 24-37), using porcine skin (thawed porcine ear skin, full thickness, shaved by clipper, purchased from Lahav (Israel)). Briefly, skin pieces were placed on the donor cells and left to warm for 15 minutes. The receiver cell was filled with 30% hydroethanolic solution (Carlo Erba, France). Composition samples (25 μl) were then applied on the epidermal side of the skin, under non-occlusive conditions for one hour, in a dark room. Blank skin sample served as control, in order to rule-out auto-fluorescence of the skin tissue. After 1 hour, the skin was removed from the cell and its surface was carefully washed twice with DDW (600 μL) and cleared using Kimwipes (Kimberly-Clark, Canada). The permeation area (0.64 mm²) was then cut using a surgical blade (Swan-Morton, England) and mounted between two cover slips of 24×60 mm for microscopic examination. Skin samples were analyzed using confocal laser scanning microscopy as detailed below.

### Scanning Electron Microscopy (SEM) and Sample Preparation

SEM samples were prepared as follows. Samples (10 μl) of vesicle compositions or 10-fold dilutions thereof (dilution with EtOH:DDW at a weight ratio of 70:17.5) were spread on 12 mm plastic coverslips (Paul Marienfeld) that were previously coated with Poly-L-Lysine. The coated coverslips were dried for 24 hours at room temperature. The coated coverslips samples were then further coated with Palladium by a sputter coater (SC7620, Quorum, UK) and then examined by SEM (FEI Quantal 200 SEM) at acceleration voltages of 5 or 10 kV.

### Transmission Electron Microscopy (TEM) and Sample Preparation

Terpenosome compositions containing geraniol were 1:10 diluted (with EtOH:DDW at 70:17.5). Samples (3 l) were placed on a 400 mesh carbon coated grid (EMS, USA) and stained with 1% aqueous solution of phosphotungstic acid (PTA) for 20 seconds, dried for 20 minutes at room temperature, then visualized using Philips TECHNAI CM 120 (Eindhoven, Netherland).

### Dynamic Light Scattering (DLS) and Sample Preparation

The mean size distribution of the vesicles in various terpenosome compositions was examined by dynamic light scattering (DLS), Malvern Zetasizer-nano, ZEN 3600, Malvern Instruments, UK. Sample preparation was as follows. Immediately before the measurements, the compositions were diluted with double distilled water at a ratio 1:100 and filtered using Bulk GHP Acrodisc® 13 mm Syringe Filters, 0.45 μm GHP membrane, Pall Corporation, USA. The apparatus was set to measure each sample three times at 25 °C. The duration and the set position of each measurement were automatically fixed by the apparatus. The test was performed at angel of 1730 to measure the size distribution by intensity MEAN±SD.

### Confocal Laser Scanning Microscopy (CLSM) and Sample Preparation

Skin samples were scanned using a confocal laser scanning microscope (CLSM, Zeiss LSM 710 laser scanning microscopy system, Zeiss, Germany) at a stack scanning mode, 1%-2% laser intensity, 10 m increments through the z-axis with an air-plane ×10 objective lens and 488 nm excitation wavelength. During the microscopic examination, each skin sample was divided to 5×5 tiles and micrographic images were obtained. The fluorescence intensity (arbitrary units) was assessed using the ImageJ software.

Assessing the Analgesic Effect of CBD Administered from Various Systems Using an Animal Pain Model

The acetic acid-induced abdominal writhing test was used for assessing the analgesic effect of CBD administered from various systems. Generally, pain was induced by an injection of acetic acid (acting as an irritant) into the peritoneal cavity of mice. When animals are intraperitoneally injected with acetic acid, a painful reaction and acute inflammation emerge in the peritoneal area. The animals react with a characteristic stretching behavior which is called writhing, as defined below. Analgesic activity of the test compound is inferred from a decrease in the frequency of writhes (i.e., of the writhing episodes).

Animals were randomly and equally divided into groups, according to the number of tests conducted. One day before the experiment, 1.5*1.5 cm² of the dorsal skin area of the animals was clipped. On the day of the experiment, the animals in the test groups were anesthetized shortly with Isoflurane® and treated with 100 mg/kg CBD in the various tested compositions. The systems were applied topically and occlusively or non-occlusively to the shaved area. The animals were anesthetized again and injected intraperitoneally with acetic acid (0.6% v/v) at a dose of (10 ml/kg) at predetermined time points after administration of CBD. A further group (n=7) served as untreated control, namely, animals in this group were anesthetized with Isoflurane® and injected with acetic acid at the same dose without receiving any drug treatment.

The number of writhing episodes was recorded by counting the number of writhes starting from five (5) minutes after acetic acid administration, for a period of 20 minutes. Writhes are indicated by the abdominal constriction and stretching of at least one hind limb. The analgesic effect of each treatment was expressed by the Maximum Possible Effect (MPE %) of the treatments, which is directly related to the efficiency of the treatment, and is calculated according to the following equation:

MPE %=[Mean of writhing in control group−number of writhing in each mouse in treated group]/[Mean of writhing in control group]*100

### Mice, Growth and Maintenance

Mice were housed under standard conditions of light and temperature in plastic cages in the specific-pathogen unit (SPF) of the pharmacy school at the Hebrew University. Animals were provided with unlimited access to water and food, with being individually inserted in separated cages with smooth flat floor.

### Examples 1-7

### Preparation of Limonene-Based Terpenosomes Comprising Cannabinoids

Limonene-based compositions (terpenosomes), including a terpenosome comprising Cannabidiol (CBD) were prepared as generally detailed above and as set out in Ex. 1 to Ex. 5 listed in Table 5. Briefly, limonene was dissolved in EtOH, glycyrrhizin was then added, through mixing using an overhead stirrer at 500 RPM. Next, when present, CBD was added and mixed well with the other components. Finally, DDW was gradually added through mixing. The weight per weight percentages (% w/w) of all ingredients are indicated in Table 5 below. The compositions listed in Ex. 6 and Ex. 7 below are prepared in a similar manner.

TABLE 5
Limonene-based terpenosomes comprising cannabinoids
Ingredients
% w/w

| | Ex. 1 | Ex. 2 | Ex. 3 | Ex. 4 | Ex. 5 | Ex. 6 | Ex. 7 |
|---|---|---|---|---|---|---|---|
| Limonene | 0.1 | 0.1 | 0.3 | 0.3 | 5 | 0.1 | 0.3 |
| Glycyrrhizin | 0.2 | 0.5 | 1 | 1 | 2 | 1 | 1 |
| EtOH | 15 | 15 | 30 | 30 | 74 | 30 | 30 |
| CBD | — | — | — | 0.3 | — | — | 0.1 |
| THC | — | — | — | — | — | 0.1 | — |
| CBN | — | — | — | — | — | — | 0.1 |
| CBG | — | — | — | — | — | — | 0.1 |
| DDW | 84.7 | 84.4 | 68.7 | 68.4 | 19 | 68.7 | 68.4 |

### Examples 8-13

### Preparation of Geraniol-Based Terpenosomes Comprising Cannabinoids

Geraniol-based compositions (terpenosomes), including a terpenosome comprising CBD were prepared as generally detailed above and as set out in Ex. 8 to Ex. 12 listed in Table 6. Briefly, glycyrrhizin was first dissolved in EtOH and geraniol was then added through mixing, using an overhead stirrer at 500 RPM. When present, CBD was next added and mixed well with the other components (Ex. 11 below). Finally, DDW was gradually added through mixing. Weight per weight percentages (% w/w) of all ingredients are indicated in Table 6 below. The composition listed in Ex. 13 is prepared in a similar manner.

TABLE 6
Geraniol-based terpenosomes comprising cannabinoids
Ingredients
% w/w

| | Ex. 8 | Ex. 9 | Ex. 10 | Ex. 11 | Ex. 12 | Ex. 13 |
|---|---|---|---|---|---|---|
| Geraniol | 0.1 | 0.1 | 0.3 | 0.3 | 10 | 0.3 |
| Glycyrrhizin | 1 | 0.4 | 1 | 1 | 2 | 1 |
| EtOH | 15 | 15 | 30 | 30 | 70 | 30 |
| CBD | — | — | — | 0.3 | — | 0.2 |
| THC | — | — | — | — | — | 0.1 |
| DDW | 83.9 | 84.5 | 68.7 | 68.4 | 18 | 68.4 |

### Example 14

### Visualization of Terpene-Based Vesicles (Terpenosomes) by Scanning Electronic Microscopy (SEM)

In order to characterize the microscopic structure of the terpenosomes prepared as described above, an exemplary terpenosome composition prepared according to Example 12 (as set out in Table 6 above) was visualized using scanning electron microscopy (SEM) as detailed above. A representative SEM micrograph of vesicles formed by the composition of Example 12 is shown in FIG. 1.

### Examples 15-20

### Preparation of Menthol-Based Terpenosomes Comprising Cannabinoids

Menthol-based compositions (terpenosomes) including a terpenosome comprising CBD were prepared as generally detailed above and as set out in Ex. 15 to Ex. 19 below (Table 7). Briefly, menthol was first dissolved in EtOH and glycyrrhizin was then added to the mixture through mixing, using an overhead stirrer at 500 RPM. When present, CBD was next added and mixed well with the other components. Finally, DDW was gradually added through mixing. Weight per weight percentages (% w/w) of all ingredients are indicated in Table 7. The composition listed in Ex. 20 below is prepared in a similar manner.

TABLE 7
Menthol-based terpenosomes comprising cannabinoids
Ingredients
% w/w

| | Ex. 15 | Ex. 16 | Ex. 17 | Ex. 18 | Ex. 19 | Ex. 20 |
|---|---|---|---|---|---|---|
| Menthol | 0.1 | 0.1 | 0.3 | 0.3 | 10 | 0.3 |
| Glycyrrhizin | 1 | 0.5 | 1 | 1 | 2 | 1 |
| EtOH | 15 | 15 | 30 | 30 | 70 | 30 |
| CBD | — | — | — | 0.3 | — | 0.2 |
| THC | — | — | — | — | — | 0.1 |
| DDW | 83.9 | 84.4 | 68.7 | 68.4 | 18 | 68.4 |

### Examples 21-26

### Preparation of Eucalyptol-Based Terpenosomes Comprising Cannabinoids

Eucalyptol-based terpenosome compositions including terpenosome comprising CBD were prepared as generally detailed above and as set out in Ex. 21 to Ex. 25 listed in Table 8. Briefly, Eucalyptol was first dissolved in absolute ethanol and glycyrrhizin was then added to the mixture through mixing, using an overhead stirrer at 500 RPM. When present, CBD was next added and mixed well with the other components. Finally, DDW was gradually added through mixing. Weight per weight percentages (% w/w) of all ingredients are indicated in Table 8 below. The composition listed in Ex. 26 below is prepared in a similar manner.

TABLE 8
Eucalyptol-based terpenosomes comprising cannabinoids
Ingredients
% w/w

| | Ex. 21 | Ex. 22 | Ex. 23 | Ex. 24 | Ex. 25 | Ex. 26 |
|---|---|---|---|---|---|---|
| Eucalyptol | 0.1 | 0.1 | 0.3 | 0.3 | 10 | 0.3 |
| Glycyrrhizin | 0.2 | 0.5 | 1 | 1 | 2 | 1 |
| EtOH | 15 | 15 | 30 | 30 | 70 | 30 |
| CBD | — | — | — | 0.3 | — | 0.2 |
| THC | — | — | — | — | — | 0.1 |
| DDW | 84.7 | 84.4 | 68.7 | 68.4 | 18 | 68.4 |

### Examples 27-33

### Preparation of Limonene-Based Terpenosomes Comprising Cannabinoids and Additional Components

Next, limonene-based terpenosomes were prepared with the addition of various additives, as set out in Ex. 28-30 and 32-33 in Table 9. First, cetyl alcohol and limonene were dissolved in absolute ethanol or 70% ethanol in a closed glass vessel using an overhead stirrer at 500 RPM. For preparing a composition comprising isopropanol (Ex. 27 in Table 9), isopropanol is also added at this stage, while stirring. Glycyrrhizin was next added through mixing, which was continued until glycyrrhizin was completely dissolved. DDW was then slowly and gradually added through mixing, which was continued for further five minutes. When present, MgCl₂ was then added and the mixing continued for additional five minutes. Finally, when present, CBD was added and mixed well using Vortex (Vortex2 Genie, MRC LTD, Israel) until homogenous appearance of the compositions was obtained (Ex. 33). Weight per weight percentages (% w/w) of all ingredients are indicated in Table 9. The composition of Ex. 31 is prepared in a similar manner.

TABLE 9
Limonene-based terpenosomes comprising cannabinoids and additional components
Ingredients
% w/w

| | Ex. 27 | Ex. 28 | Ex. 29 | Ex. 30 | Ex. 31 | Ex. 32 | Ex. 33 |
|---|---|---|---|---|---|---|---|
| Limonene | 5 | 2 | 2 | 5 | 4.75 | 1.75 | 4.75 |
| Glycyrrhizin | 2 | 2 | 1 | 2 | 1.9 | 1.9 | 0.9 |
| Cetyl alcohol | 0.4 | 0.3 | 0.5 | 0.7 | 0.475 | 0.275 | 0.475 |
| EtOH | 64 | — | 74.9 | 73.8 | 70.3 | 73.3 | 70.11 |
| Ethanol 70% | — | 76.2 | — | — | — | — | — |
| Isopropanol | 10 | — | — | — | — | — | — |
| MgCl₂ | — | — | 0.1 | 0.2 | — | — | 0.19 |
| CBD | — | — | — | — | 4.5 | — | 5 |
| CBN | — | — | — | — | 0.3 | — | — |
| THC | — | — | — | — | 0.1 | — | — |
| CBG | — | — | — | — | 0.1 | — | — |
| DDW | 18.6 | 19.5 | 19.3 | 18.3 | 17.575 | 17.775 | 18.575 |

### Examples 34-40

### Preparation of Geraniol-Based Terpenosomes Comprising CBD and Additional Components

Further to the above preparations, geraniol-based terpenosomes were prepared with the addition of various additives, as set out in Ex. 34, 36-37 and 39-40 (Table 10 below). Briefly, Geraniol and glyccirrhizin were first dissolved in EtOH using an overhead stirrer, at 500 RPM. Cetyl alcohol was then added and mixed well until completely dissolved. When present, isopropanol is also added at this stage, while stirring (for preparing the compositions of Ex. 35 and Ex. 38). DDW was then slowly and gradually added through mixing, which was continued for further five minutes. When present, MgCl₂ was then added and the mixing continued for additional five minutes. CBD was added and mixed well with Vortex, as detailed above (Ex. 39). When present, hydroxypropylmethyl cellulose was then added and mixed well. Weight per weight percentages (% w/w) of all ingredients are indicated in Table 10 below.

TABLE 10
Geraniol-based terpenosomes comprising cannabinoids and additional components
Ingredients
% w/w

| | Ex. 34 | Ex. 35 | Ex. 36 | Ex. 37 | Ex. 38 | Ex. 39 | Ex. 40 |
|---|---|---|---|---|---|---|---|
| Geraniol | 10 | 5 | 2 | 10 | 10 | 9.5 | 0.3 |
| Glycyrrhizin | 2 | 2 | 1 | 2 | 0.5 | 1.9 | 0.5 |
| Cetyl alcohol | 0.5 | 0.5 | 0.2 | 0.5 | 0.5 | 0.475 | — |
| EtOH | 70 | 64 | 60 | 69.8 | 59.9 | 66.5 | 40 |
| Isopropanol | — | 10 | — | — | 10 | — | — |
| MgCl₂ | — | — | — | 0.2 | 0.1 | 0.19 | — |
| CBD | — | — | — | — | — | 5 | 0.4 |
| Hydroxypropylmethyl | — | — | — | — | — | — | 0.7 |
| cellulose | | | | | | | |
| DDW | 17.5 | 18.5 | 36.8 | 17.5 | 19.0 | 16.435 | 58.1 |

### Examples 41-46

### Preparation of Menthol-Based Terpenosomes Comprising CBD and Additional Components

Menthol-based terpenosomes were also prepared with the addition of various additives, as set out in Table 11 below, as generally described above. Briefly, menthol and cetyl alcohol were dissolved in EtOH or ethanol 96% in a closed glass vessel using an overhead stirrer at at 500 RPM. Glycyrrhizin was added through mixing, which was continued until the glycyrrhizin completely dissolved. DDW was then slowly and gradually added through mixing. When present, MgCl₂ was next added and the mixing continued for five minutes. CBD was added and mixed well with Vortex until homogenous appearance of the composition was obtained (i.e., Ex. 45 and Ex. 46). When present, propylene glycol was then added and mixed well. Weight per weight percentages (% w/w) of all ingredients are indicated in Table 11.

TABLE 11
Menthol-based terpenosomes comprising
cannabinoids and additional components
Ingredients
% w/w

| | Ex. 41 | Ex. 42 | Ex. 43 | Ex. 44 | Ex. 45 | Ex. 46 |
|---|---|---|---|---|---|---|
| Menthol | 10 | 5 | 2 | 10 | 9.5 | 1 |
| Glycyrrhizin | 2 | 2 | 1 | 2 | 3 | 1.9 |
| Cetyl alcohol | 0.5 | 1 | 0.5 | 0.5 | 0.475 | 0.475 |
| EtOH | 70 | 74 | 52 | — | 66.5 | 70.31 |
| Ethanol 96% | — | — | — | 69.8 | — | — |
| Propylene | — | — | 22.2 | — | — | — |
| glycol | | | | | | |
| MgCl₂ | — | — | — | 0.2 | — | 0.19 |
| CBD | — | — | — | — | 5 | 5 |
| DDW | 17.5 | 18.0 | 22.3 | 17.5 | 15.525 | 21.125 |

### Examples 47-52

### Preparation of Eucalyptol-Based Terpenosomes Comprising CBD and Additional Components

Eucalyptol-based terpenosomes were next prepared with the addition of various additives, as set out in Ex. 47-48 and 50-52 in Table 12 below. Briefly, glyccirhizin was first dissolved in EtOH using an overhead stirrer at 500 RPM. Cetyl alcohol and eucalyptol were then added and mixed well until completely dissolved. When present, isopropanol is also added at this stage, while stirring (for preparing the composition of Ex. 49). DDW was then slowly and gradually added through mixing which was continued for further five minutes. When present, MgCl₂ was then added and the mixing continued for additional five minutes. CBD was added and mixed well, with Vortex (i.e., Ex. 51 and Ex. 52). Weight per weight percentages (% w/w) of all ingredients are indicated in Table 12.

TABLE 12
Eucalyptol-based terpenosomes comprising
cannabinoids and additional components
Ingredients
% w/w

| | Ex. 47 | Ex. 48 | Ex. 49 | Ex. 50 | Ex. 51 | Ex. 52 |
|---|---|---|---|---|---|---|
| Eucalyptol | 10 | 5 | 2 | 10 | 1 | 9.5 |
| Glycyrrhizin | 2 | 2 | 1 | 5 | 1.9 | 1.9 |
| Cetyl alcohol | 0.5 | 0.2 | 0.3 | 0.5 | 0.325 | 0.475 |
| EtOH | 70 | 74.3 | 37.4 | 71.8 | 70.35 | 66.31 |
| Isopropanol | — | — | 30 | — | — | — |
| MgCl₂ | — | — | — | 0.2 | — | 0.19 |
| CBD | — | — | — | — | 5 | 5 |
| DDW | 17.5 | 18.5 | 29.3 | 12.5 | 21.325 | 16.625 |

### Examples 53-60

### Preparation of Terpenosomes Comprising Multiple Terpenes and CBD

Terpenosome compositions comprising multiple terpenes and CBD were next prepared, as set out in Table 13 below and as generally described above. Briefly, cetyl alcohol was first mixed with the terpenes using an overhead stirrer at 500 RPM. EtOH was then added and mixed for one minute. Mixing speed was lowered to 300 RPM and glycyrrhizin was added through mixing, which was continued until the glycyrrhizin completely dissolved. DDW was then slowly and gradually added through mixing, which was continued for further five minutes. MgCl₂ was next added and the mixing continued for additional five minutes. CBD was next added and mixed well with the other components, by Vortex. When present, propylene glycol was then added and mixed well. The weight per weight percentages (% w/w) of all ingredients are indicated in Table 13.

TABLE 13
Terpenosomes comprising multiple terpenes and CBD
Ingredients
% w/w

| | Ex. 53 | Ex. 54 | Ex. 55 | Ex. 56 | Ex. 57 | Ex. 58 | Ex. 59 | Ex. 60 |
|---|---|---|---|---|---|---|---|---|
| Limonene | 1.25 | 1.25 | 1.19 | 1.19 | 1.25 | 1.25 | 1.19 | 1.19 |
| Geraniol | 1.25 | 1.25 | 1.19 | 1.19 | 1.25 | 1.25 | 1.19 | 1.19 |
| Menthol | 1.25 | 1.25 | 1.19 | 1.19 | 1.25 | 1.25 | 1.19 | 1.19 |
| Eucalyptol | 1.25 | 1.25 | 1.9 | 1.9 | 1.25 | 1.25 | 1.19 | 1.19 |
| Glycyrrhizin | 1 | 2 | 1.9 | 1.9 | 1 | 3 | 2.9 | 1.9 |
| Cetyl alcohol | — | — | — | — | 0.5 | 0.3 | — | 0.5 |
| EtOH | 70 | 70 | 66.5 | 66.5 | 70 | 69 | 66.5 | 66.5 |
| Propylene glycol | 1 | 5 | — | 4.75 | | 5 | — | 4.75 |
| MgCl₂ | — | — | — | — | 0.2 | — | — | — |
| CBD | — | — | 5 | 5 | — | — | 3 | — |
| DDW | 24 | 18 | 21.1 | 16.38 | 23.3 | 17.7 | 22.84 | 21.59 |

### Examples 61-71

### Preparation of Terpenosomes Comprising Multiple Terpenes and CBD in the Presence of 1,3-Propanediol

Preparation of terpenosomes comprising multiple terpenes and CBD in the presence of 1,3-Propanediol was as detailed in Table 14 below. Briefly, 1,3-Propanediol was first mixed with EtOH. Then the terpene mixture and CBD, when present, were added while using an overhead stirrer at a speed of 700 RPM. Glycyrrhizin was added through mixing at the same speed, which was continued until the glycyrrhizin completely dissolved. DDW was then added slowly and gradually through mixing, which was continued for further five minutes. Weight per weight percentages (% w/w) of all ingredients are indicated in Table 14.

TABLE 14
Terpenosomes comprising multiple terpenes and CBD in the presence of
Ingredients
% w/w

| | Ex. | Ex. | Ex. | Ex. | Ex. | Ex. | Ex. | Ex. | Ex. | Ex. | Ex. |
|---|---|---|---|---|---|---|---|---|---|---|---|
| | 61 | 62 | 63 | 64 | 65 | 66 | 67 | 68 | 69 | 70 | 71 |
| Limonene | 10 | — | 1.25 | 1.25 | 0.5 | 0.25 | — | 0.25 | 0.25 | 0.125 | 0.125 |
| Geraniol | — | 10 | 1.25 | 1.25 | 0.5 | 0.25 | — | 0.25 | 0.25 | 0.125 | 0.125 |
| Menthol | — | — | 1.25 | 1.25 | 0.5 | 0.25 | 0.5 | 0.25 | 0.25 | 0.125 | 0.125 |
| Eucalyptol | — | — | 1.25 | 1.25 | 0.5 | 0.25 | 0.5 | 0.25 | 0.25 | 0.125 | 0.125 |
| Glycyrrhizin | 2 | 2 | 1 | 2 | 2 | 1 | 3 | 2 | 0.5 | 2 | 1.5 |
| 1,3-Propanediol | 15 | 30 | 40 | 40 | 50 | 50 | 40 | 50 | 50 | 50 | 50 |
| EtOH | 55 | 40 | 34 | 34 | 20 | 20 | 30 | 20 | 20 | 21 | 21 |
| CBD | — | — | — | 1 | — | — | 0.5 | 0.5 | 1 | — | 0.5 |
| DDW | 18 | 18 | 20 | 18 | 26 | 28 | 25.5 | 26.5 | 27.5 | 26.5 | 26.5 |

### Examples 72-77

### Preparation of Caryophyllene Oxide-Based Terpenosomes Comprising Cannabinoids

Caryophyllene oxide-based compositions (terpenosomes), including a terpenosome comprising CBD are prepared as generally detailed above and as set out in Table 15 below. Briefly, Caryophyllene oxide is first dissolved in EtOH and glycyrrhizin is then added to the mixture through mixing, using an overhead stirrer at 500 RPM. Cannabinoids and cetyl alcohol, if present, are next added and mixed well with the other components. Finally, DDW is gradually added through mixing. Weight per weight percentages (% w/w) of all ingredients are indicated in Table 15 below.

TABLE 15
Caryophyllene oxide-based terpenosomes comprising cannabinoids
Ingredients
% w/w

| | Ex. 72 | Ex. 73 | Ex. 74 | Ex. 75 | Ex. 76 | Ex. 77 |
|---|---|---|---|---|---|---|
| Caryophyllene | 0.1 | 10 | 0.3 | 0.3 | 5 | 0.3 |
| oxide | | | | | | |
| Cetyl alcohol | — | 0.5 | — | — | — | 0.2 |
| Glycyrrhizin | 1 | 2 | 1 | 3 | 2 | 1 |
| EtOH | 15 | 69.5 | 32 | 30 | 70 | 30 |
| CBD | — | — | — | 0.3 | 4.9 | 0.2 |
| THC | — | — | — | — | 0.1 | — |
| DDW | 83.9 | 18 | 66.7 | 66.4 | 18 | 68.3 |

### Examples 78-83

### Preparation of Alpha-Bisabolol-Based Terpenosomes Comprising CBD

Alpha-Bisabolol-based compositions (terpenosomes) comprising CBD as set out in the following Table (16) are prepared as generally detailed above. Briefly, Alpha-Bisabolol is first dissolved in EtOH and glycyrrhizin is then added to the mixture through mixing, using an overhead stirrer at 500 RPM. CBD and cetyl alcohol, if present, are next added and mixed well with the other components. Finally, DDW is gradually added through mixing. Magnesium salt, if present, is dissolved in the DDW. Weight per weight percentages (% w/w) of all ingredients are indicated in Table 16 below.

TABLE 16
Alpha-Bisabolol-based terpenosomes comprising cannabinoids
Ingredients
% w/w

| | Ex. 78 | Ex. 79 | Ex. 80 | Ex. 81 | Ex. 82 | Ex. 83 |
|---|---|---|---|---|---|---|
| Alpha- | 0.1 | 0.1 | 0.3 | 0.3 | 10 | 0.3 |
| Bisabolol | | | | | | |
| Cetyl alcohol | — | — | — | — | 0.4 | — |
| MgCl₂ | 0.1 | — | — | — | — | — |
| Glycyrrhizin | 1 | 0.5 | 1 | 1.2 | 2 | 1 |
| EtOH | 17 | 15 | 30 | 30 | 68 | 30 |
| CBD | — | — | — | 0.3 | 3 | 0.2 |
| DDW | 81.8 | 84.4 | 68.7 | 68.2 | 16.6 | 68.5 |

### Examples 84-91

### Preparation of Terpenosome Compositions for the Treatment of Acne

Preparation of terpenosome compositions for the treatment of Acne is as follows and as set out in Table 17. Propandiol is first mixed with EtOH. Then the terpene mixture and CBD, when present, are dissolved in the solution using an overhead stirrer as detailed above, at a speed of 700 RPM. Glycyrrhizin is then added through mixing at the same speed, which was continued until the glycyrrhizin completely dissolves. DDW is next slowly and gradually added, through mixing, which is continued for further five minutes. The additional active ingredients (listed in Table 17), are added during the different stages of the preparation such that lipophilic active agents are mixed with the organic phase and hydrophilic (water-soluble) molecules (agents) are first mixed with water and then gradually added to the organic phase. Weight per weight percentages (% w/w) of all ingredients are indicated in Table 17.

TABLE 17
Terpenosomes comprising multiple terpenes, CBD and additional active
agents for the treatment of Acne
Ingredients
% w/w

| | Ex. 84 | Ex. 85 | Ex. 86 | Ex. 87 | Ex. 88 | Ex. 89 | Ex. 90 | Ex. 91 |
|---|---|---|---|---|---|---|---|---|
| Limonene | 0.5 | 0.5 | 0.25 | 1.25 | 2.5 | 0.5 | 0.125 | 0.25 |
| Geraniol | — | 0.5 | 0.25 | 1.25 | — | — | — | 0.25 |
| Menthol | 0.5 | 0.5 | 0.25 | 1.25 | 2.5 | 0.5 | 0.125 | 0.25 |
| Eucalyptol | — | 0.5 | 0.25 | 1.25 | — | — | — | 0.25 |
| Glycyrrhizin | 1 | 2 | 2 | 2 | 0.5 | 1 | 1 | 2 |
| 1,3-Propanediol | 35 | 30 | 40 | 20 | 20 | 35 | 40 | 30 |
| EtOH | 30 | 45.9 | 20 | 48 | 50 | 35 | 15 | 29.9 |
| CBD | — | 3 | — | 1 | 0.3 | 0.2 | — | 0.5 |
| Salicylic acid | 2 | — | — | 5 | — | 2 | — | 0.1 |
| Tretinoin | — | 0.05 | — | — | — | — | — | — |
| Resorcinol | — | — | 1.25 | — | — | — | — | — |
| Retinol | — | — | — | 1 | — | — | — | — |
| Adapalene | — | — | — | — | 1 | — | — | — |
| Niacinamide | — | — | — | — | — | — | 1 | — |
| Azelaic acid | — | — | — | — | — | 5 | — | 10 |
| DDW | 31 | 17.05 | 35.75 | 18 | 23.2 | 20.8 | 42.75 | 26.5 |

### Examples 92-99

### Preparation of Terpenosome Compositions for Skin Exfoliation

Terpenosome compositions for skin exfoliation are prepared as follows and as set out in Table 18 below. Propandiol is first mixed with EtOH. Then the terpene mixture and CBD, when present, are dissolved in this solution, using an overhead stirrer at a speed of 700 RPM. Glycyrrhizin is then added through mixing at the same speed, which is continued until the glycyrrhizin completely dissolves. DDW is next slowly and gradually added through mixing, which is continued for further five minutes. Further active ingredients are added during the different stages of the preparation process, as detailed above. Weight per weight percentages (% w/w) of all ingredients are indicated in Table 18.

TABLE 18
Terpenosomes compositions for skin exfoliation
Ingredients
% w/w

| | Ex. 92 | Ex. 93 | Ex. 94 | Ex. 95 | Ex. 96 | Ex. 97 | Ex. 98 | Ex. 99 |
|---|---|---|---|---|---|---|---|---|
| Limonene | 0.5 | 0.5 | 0.25 | 0.5 | — | 0.5 | 0.125 | 0.25 |
| Geraniol | — | 0.5 | 0.25 | — | — | — | — | 0.25 |
| Menthol | 0.5 | 0.5 | 0.25 | — | 2.5 | 0.5 | 0.125 | 0.25 |
| Eucalyptol | — | 0.5 | 0.25 | — | — | — | — | 0.25 |
| Glycyrrhizin | 1 | 2 | 2 | 2 | 0.5 | 1 | 1 | 2 |
| 1,3-Propanediol | 10 | 15 | 30 | 30 | 20 | | 40 | 30 |
| EtOH | 50 | 48.9 | 20 | 53 | 50 | 40 | 15 | 37 |
| CBD | — | — | — | — | 0.3 | 0.2 | 0.1 | 0.5 |
| Salicylic acid | 5 | — | 1 | — | 5 | — | 1 | — |
| Mandelic acid | — | — | — | — | — | 3 | — | — |
| Lactic acid | — | — | — | 1 | — | — | — | — |
| Glycolic acid | — | 3 | — | — | — | — | — | 3 |
| DDW | 33 | 29.1 | 46 | 13.5 | 21.7 | 54.8 | 42.65 | 26.5 |

### Examples 100-108

### Preparation of Terpenosome Compositions for Skin Anti-Aging

Preparation of terpenosome compositions for skin anti-aging is as follows and as set out in Table 19 below. Propandiol is first mixed with EtOH. Then, the terpene mixture and CBD, when present, are dissolved in this solution using an overhead stirrer, at 700 RPM. Glycyrrhizin is then added through mixing at the same speed, which is continued until the glycyrrhizin completely dissolved. DDW is then slowly and gradually added through mixing, which is continued for further five minutes. The further active ingredients are added during the different stages of the preparation process, as detailed above. Weight per weight percentages (% w/w) of all ingredients are indicated in Table 19.

TABLE 19
Terpenosomes compositions for skin anti-aging
Ingredients
% w/w

| | Ex. | Ex. | Ex | Ex. | Ex. | Ex. | Ex. | Ex. | Ex. |
|---|---|---|---|---|---|---|---|---|---|
| | 100 | 101 | 102 | 103 | 104 | 105 | 106 | 107 | 108 |
| Limonene | 1 | | 1.25 | 1.25 | 0.5 | 0.25 | 0.5 | 0.25 | 0.5 |
| Linalool | 1 | 0.5 | 1.25 | — | 0.5 | 0.25 | 0.5 | — | — |
| Citronellol | 0.2 | 0.5 | — | — | 0.5 | — | 0.5 | 0.25 | — |
| Menthol | — | — | 1.25 | 1.25 | — | 0.25 | — | — | 0.5 |
| Glycyrrhizin | 2 | 2 | 2 | 2 | 1 | 2 | 2 | 2 | 2 |
| 1,3-Propanediol | 64 | 30 | 40 | 40 | 60.5 | 50 | 40 | 40 | 50 |
| EtOH | 18.8 | 40 | 34 | 34 | 20 | 20 | 30 | 20 | 20 |
| CBD | 2 | — | 0.1 | 1 | 5 | — | 0.05 | 0.5 | — |
| Vitamin C | 10 | — | — | — | 10 | — | — | — | — |
| Hyaluronic acid | — | 0.2 | 0.5 | | — | — | — | — | — |
| Retinol | — | — | — | 0.25 | — | — | — | — | — |
| Alpha arbutin 2% | — | — | — | — | 2 | — | — | — | 2 |
| Alpha Lipoic Acid | 1 | — | — | — | — | — | — | 5 | — |
| Caffeine | — | — | — | — | — | 3 | 3 | — | — |
| DDW | — | 26.8 | 19.65 | 20.25 | — | 24.25 | 23.45 | 32 | 25 |

### Examples 109-117

### Preparation of Terpenosome Compositions for Treatment of Skin Wrinkles and Texture Irregularities

Preparation of terpenosome compositions for treatment of skin wrinkles and texture irregularities is as follows and as set out in Table 20 below. Propandiol is first mixed with EtOH. Then the terpene mixture and CBD, when present, are dissolved in this solution using an overhead stirrer, as detailed above, at a speed of 700 RPM. Glycyrrhizin is next added, through mixing at the same rpm speed, which is continued until the glycyrrhizin completely dissolved. DDW is then slowly and gradually added through mixing, which is continued for further five minutes. The other active ingredients as listed in Table 20 are added during the different stages of the preparation process. Weight per weight percentages (% w/w) of all ingredients are indicated in Table 20.

TABLE 20
Terpenosome compositions for treatment of
skin wrinkles and texture irregularities
Ingredients
% w/w

| | Ex. | Ex. | Ex. | Ex. | Ex. | Ex. | Ex. | Ex. | Ex. |
|---|---|---|---|---|---|---|---|---|---|
| | 109 | 110 | 111 | 112 | 113 | 114 | 115 | 116 | 117 |
| Limonene | 0.1 | — | — | 1.25 | 0.15 | — | 0.5 | 0.25 | 0.1 |
| Geraniol | 0.1 | 0.5 | 0.25 | — | 0.15 | 0.25 | 0.5 | — | — |
| Menthol | 0.1 | 0.5 | — | — | 0.15 | — | 0.5 | 0.25 | — |
| Linalool | — | — | 0.25 | 1.25 | — | 0.25 | — | — | 0.3 |
| Eucalyptol | — | — | — | — | 0.1 | — | — | 0.1 | 0.1 |
| Glycyrrhizin | 2 | 2 | 2 | 2 | 2 | 2 | 2 | 2 | 2 |
| 1,3-Propanediol | 45 | 40 | 40 | 40 | 50 | 45 | 40 | 50 | 50 |
| EtOH | 15 | 20 | 15 | 34 | 20 | 15 | 40 | 20 | 20 |
| CBD | — | — | — | — | — | 0.02 | 3 | 0.1 | 0.2 |
| Niacinamide | 2 | — | — | — | — | 1 | — | — | — |
| Retinol | — | 0.5 | — | — | — | — | 0.5 | — | — |
| Copper peptides | — | — | 1 | 1 | — | — | — | 0.8 | — |
| Hyaluronic acid | — | — | 0.2 | — | 0.5 | — | — | — | 1 |
| DDW | 35.7 | 36.5 | 41.3 | 20.5 | 26.95 | 36.48 | 14 | 26.5 | 26.3 |

### Examples 118-126

### Preparation of Terpenosome Compositions Comprising Active Agents

Further terpenosome compositions comprising various additional active agents as well as cetyl alcohol, were prepared as generally described above and as set out in Table 21 below. Briefly, terpenes were dissolved in EtOH, and mixed using an overhead stirrer at 700 RPM. When present, cetyl alcohol was added at this stage. Glycyrrhizin was then added through mixing, at the same rpm. When present, lipophilic active agents and polyols were dissolved in the organic phase as well. The mixing was continued until the glycyrrhizin completely dissolved. DDW (and if present, hydrophilic active agent which was first dissolved therein) was then slowly and gradually added through mixing which was continued for further five (5) minutes.

TABLE 21
Terpenosome compositions comprising various active agents
Ingredients
% w/w

| | Ex. | Ex. | Ex. | Ex. | Ex. | Ex. | Ex. | Ex. | Ex. |
|---|---|---|---|---|---|---|---|---|---|
| | 118 | 119 | 120 | 121 | 122 | 123 | 124 | 125 | 126 |
| Vitamin D | 1 | 1 | — | — | — | — | — | — | — |
| Lidocaine base | — | — | 5 | 5 | — | — | — | — | — |
| Melatonin | — | — | — | — | 0.5 | — | — | — | — |
| Ibuprofen | — | — | — | — | — | 10 | — | — | — |
| Vitamin E (Tocopherol | — | — | — | — | — | — | 0.5 | 1 | — |
| acetate) | | | | | | | | | |
| Vitamin C | - | — | — | — | — | — | — | — | 1 |
| Limonene | 1 | 1 | 5 | 1 | 0.25 | — | 1 | 1 | — |
| Geraniol | 1 | 1 | — | 1 | 0.25 | 2 | 1 | 1 | 1 |
| Menthol | 1 | 1 | — | 1 | 0.25 | 2 | — | 2 | — |
| Eucalyptol | 1 | 1 | 5 | 2 | 0.25 | 2 | 1 | 1 | 1 |
| Glycyrrhizin | 2 | 2 | 2.2 | 1.8 | 1.2 | 2.5 | 1 | 1 | 2 |
| 1,3-Propanediol | 20 | 20 | — | — | — | 12 | 23 | | 35 |
| Cetyl alcohol | — | 0.5 | — | 0.6 | — | — | — | 0.5 | — |
| Propylene glycol | — | — | — | — | 30 | — | — | — | — |
| EtOH | 50 | 53.5 | 70 | 70 | 55 | 50 | 58 | 74 | 40 |
| DDW | 23 | 19 | 12.8 | 17.6 | 12.3 | 19.5 | 14.5 | 18.5 | 20 |

### Example 127

### Characterizing the Terpenosome Compositions by Dynamic Light Scattering and Scanning Electron Microscopy

In order to further confirm formation of vesicles in the terpenosome compositions described above and characterize their dimensions, various terpenosome prepared as described herein, having specific compositions as listed in Table 22 below, were subjected to SEM analysis and the mean size distribution of the vesicles formed was evaluated by dynamic light scattering (DLS). Samples for SEM and DLS analysis were prepared as described above and the results obtained are listed in Table 22, below.

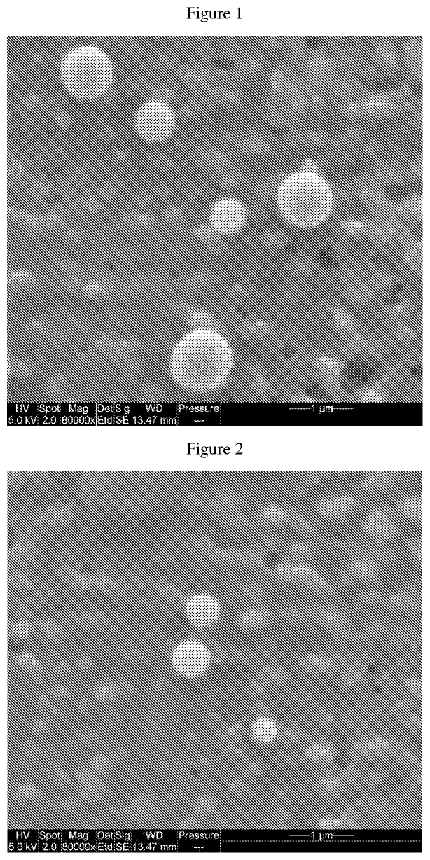

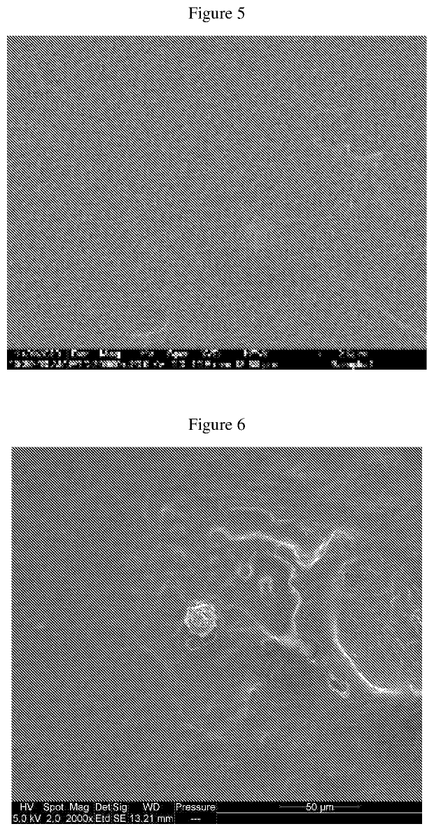

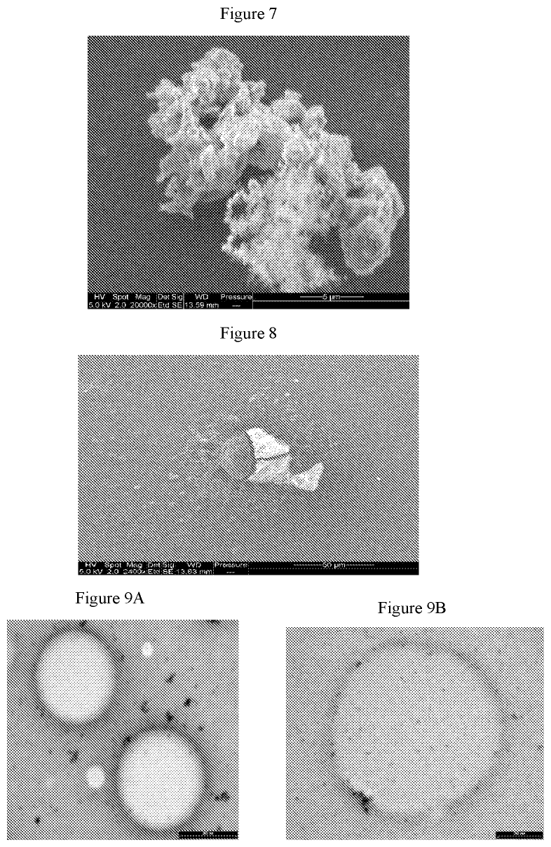

TABLE 22
Summary of the characteristics of various terpenosome systems

| | | Mean | |
|---|---|---|---|
| | | vesicular | |
| Terpenosome | | size (nm)/ | Figure number showing |
| Composition | Organoleptic | PDI (Mean ± | microscopic structure |
| (Per weight ratio) | characterization | SD) | by SEM |
| Geraniol:Glycyr- | Transparent | — | FIG. 2 |
| rhizin:EtOH:DDW | homogenous liquid | | |
| (10:2:70:17.5) | | | |
| CBD:Geraniol:Glycyr- | Yellow Transparent | — | FIG. 3 |
| rhizin:EtOH:DDW | homogenous liquid | | |
| (5:9.5:1.9:66.5:17.1) | | | |
| Cetyl | White semitransparent | 355.6 ± 131.8/ | Nanovesicles |
| alcohol:Limonene:Glycyr- | homogenous liquid | 0.772 ± | were observed |
| rhizin:EtOH:DDW | | 0.220 | |
| (0.5:10:2:70:17.5) | | | |
| Cetyl | Transparent | 309.4 ± 101.5/ | Nanovesicles |
| alcohol:Geraniol:Glycyr- | homogenous liquid | 0.479 ± | were observed |
| rhizin:EtOH:DDW | | 0.046 | |
| (0.5:10:2:70:17.5) | | | |
| Cetyl | Transparent | 121.2 ± 1.2/ | Nanovesicles |
| alcohol:Menthol:Glycyr- | homogenous liquid | 0.157 ± | were observed |
| rhizin:EtOH:DDW | | 0.019 | |
| (0.5:10:2:70:17.5) | | | |
| Cetyl | Transparent | 111.4 ± 1.0/ | FIG. 4 |
| alcohol:Eucalyptol:Glycyr- | homogenous liquid | 0.227 ± | |
| rhizin:EtOH:DDW | | 0.013 | |
| (0.5:10:2:70:17.5) | | | |

| | | | Exemplary preparation, |
|---|---|---|---|
| | | | Figure number |
| Terpenosome | | Mean | showing |
| Composition | Organoleptic | vesicular | microscopic |
| (per weight ratio) | characterization | size (nm) | structure by SEM |
| Cetyl | Yellow homogenous | 138.4 ± 0.6/ | Nanovesicles were |
| alcohol:Limonene:CBD:Glycyr- | transparent liquid | 0.49 ± | observed |
| rhizin:EtOH:DDW | | 0.004 | |
| (0.475:9.5:5:1.9:66.5:16.62) | | | |
| Cetyl | Yellow homogenous | 319.0 ± 256.1/ | Nanovesicles were |
| alcohol:Geraniol:CBD:Glycyr- | transparent liquid | 0.330 ± | observed |
| rhizin:EtOH:DDW | | 0.210 | |
| (0.475:9.5:5:1.9:66.5:16.62) | | | |
| Cetyl | Yellow homogenous | 116.7 ± 3.6/ | Nanovesicles were |
| alcohol:Menthol:CBD:Glycyr- | transparent liquid | 0.042 ± | observed |
| rhizin:EtOH:DDW | | 0.005 | |
| (0.475:9.5:5:1.9:66.5:16.62) | | | |
| Cetyl | Yellow homogenous | 132.8 ± 2.9/ | Nanovesicles were |
| alcohol:Eucalyptol:CBD:Glycyr- | transparent liquid | 0.041 ± | observed |
| rhizin:EtOH:DDW | | 0.013 | |
| (0.475:9.5:5:1.9:66.5:16.62) | | | |
| Cetyl alcohol:Geraniol:Glycyr- | Homogenous | 238.7 ± 58.2/ | Nanovesicles were |
| rhizin:EtOH:MgCl₂:DDW | transparent liquid | 0.311 ± | observed |
| (0.5:10:2:69.8:0.2:17.5) | | 0.022 | |
| Cetyl | Yellow homogenous | 169.2 ± 83.6/ | Nanovesicles were |
| alcohol:Geraniol:CBD:Glycyr- | transparent liquid | 0.253 ± | observed |
| rhizin:EtOH:MgCl₂:DDW | | 0.024 | |
| (0.475:9.5:5:1.9:66.31:0.19:16.62) | | | |
| Control lacking terpene | Transparent | — | FIG. 5 |
| Cetyl | homogenous liquid | | No vesicles were |
| alcohol:Terpene:Glycyr- | | | observed |
| rhizin:EtOH:DDW | | | |
| (0.5:0:1:70:17.5) | | | |
| Control lacking terpene | Yellow Transparent | — | FIG. 6 |
| CBD:Glycyrrhizin:EtOH:DDW | homogenous liquid | | No vesicles were |
| (5:1.9:66.5:17.1) | | | observed |
| Control lacking terpene | Yellow transparent | — | FIG. 7 |
| and glycyrrhizin | homogenous liquid | | No vesicles were |
| CBD:EtOH:DDW | | | observed |
| (5:70:17.5) | | | |
| Control lacking terpene | Yellow Transparent | — | FIG. 8 |
| and CBD | homogenous liquid | | No vesicles were |
| Glycyrrhizin:EtOH:DDW | | | observed |
| (2:70:17.5) | | | |

As clearly shown in Table 22, and for example in FIGS. 2-4, nano-vesicles (nanovesicles) were observed for a terpene-based preparation. Apparently, no vesicles were observed in the absence of a terpene (e.g., as shown in in FIG. 5 and FIG. 6).

### Example 128

### Characterizing the Terpenosome Compositions by Transmission Electron Microscopy

Transmission electron microscopy (TEM) was used to further characterize the vesicular structure of the terpenosome compositions prepared as described above. Terpenosome compositions containing geraniol, prepared as set out in Table 23 below were used for the analysis and TEM samples were prepared based thereon, as detailed above.

Briefly, compositions were prepared by mixing cetyl alcohol with geraniol in a closed glass vessel using an overhead stirrer, at a speed of 500 RPM. EtOH was then added and mixed for one minute. The mixing speed was lowered to 300 RPM, and glycyrrhizin was added through mixing, which was continued until the glycyrrhizin completely dissolved. DDW was then slowly and gradually added through mixing. MgCl₂ was then added and the mixing continued for additional five minutes. CBD was finally added and mixed well with the other components.

TABLE 23
Terpenosome compositions containing
geraniol for TEM characterization

| | Ingredients | | | |
|---|---|---|---|---|
| | | | | Ex. 128d |
| | | Ex. 128b | Ex. 128c | Geraniol, |
| | Ex. 128a | Geraniol & | Geraniol | MgCl₂ |
| | Geraniol | MgCl₂ | & CBD | & CBD |
| Ingredients | % w/w | | | |
| CBD | — | — | 5 | 5 |
| Geraniol | 10 | 10 | 9.5 | 9.5 |
| Glycyrrhizin | 2 | 2 | 1.9 | 1.9 |
| Cetyl alcohol | 0.5 | 0.5 | 0.475 | 0.475 |
| MgCl₂ | — | 0.2 | — | 0.19 |
| EtOH | 70 | 69.8 | 66.5 | 66.31 |
| DDW | 17.5 | 17.5 | 16.625 | 16.625 |

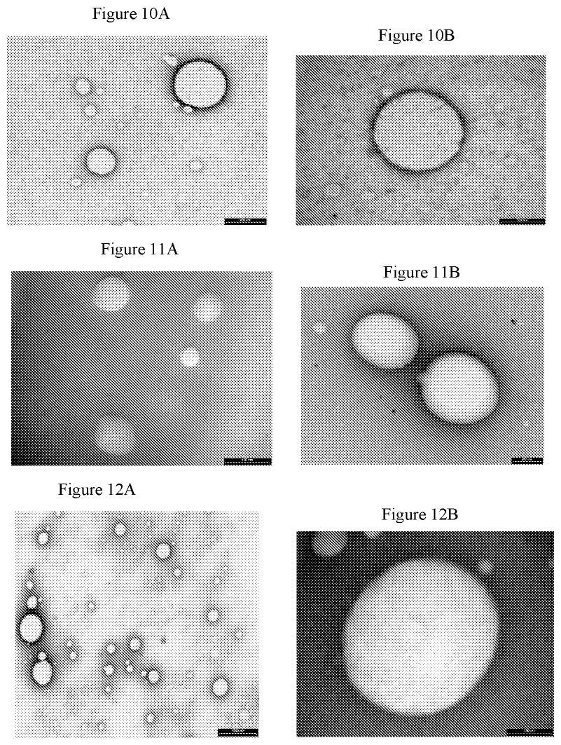

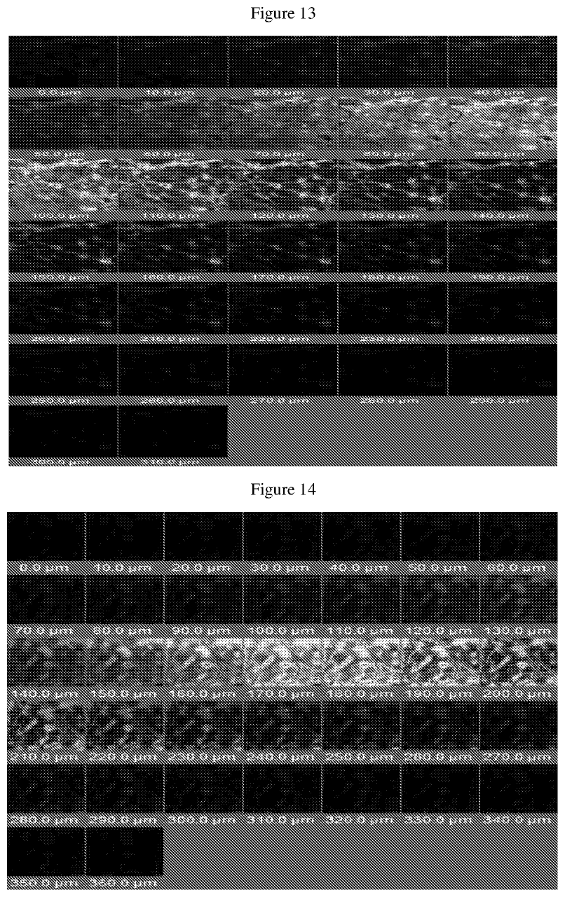

Exemplary TEM micrographs showing the vesicles obtained for the various terpenosome systems containing geraniol prepared as described above are shown in FIGS. 9 through 12. Specifically, FIG. 9A and FIG. 9B demonstrate terpenosomes containing geraniol (i.e., Ex. 128a without CBD and MgCl₂), FIG. 10A and FIG. 10B demonstrate terpenosomes containing geraniol and MgCl₂ (i.e., Ex. 128b without CBD), FIG. 11A and FIG. 11B demonstrate terpenosomes containing geraniol and CBD (i.e., Ex. 128c, without MgCl₂) and FIG. 12A and FIG. 12B demonstrate terpenosomes containing geraniol, MgCl₂ and CBD (i.e., Ex. 128d).

### Example 129

### Effect of Magnesium on Skin Penetration of Molecules Incorporated in Terpenosomes—a Confocal Laser Scanning Microscope Study

The effect of magnesium in a terpenosome composition on the skin penetration of FITC was evaluated using Franz diffusion cell and confocal laser scanning microscopy (CLSM) analysis. The lipophilic fluorescent molecule fluorescein isothiocyanate (FITC, log p 4.3) was incorporated in a limonene-based terpenosome, with and without magnesium, prepared as described above, as well as in a control composition, containing the same limonene concentration.

First, limonene-based terpenosome compositions containing FITC, in the absence of MgCl₂ (Ex. 129a) and in the presence of MgCl₂ (Ex. 129b), were prepared as described above and as set out in Table 24 below. FITC was added to the composition (two hours before starting the experiment) and mixed well with the other components. In addition, a limonene-based terpenosome composition containing FITC as described above was also prepared in the presence of MgCl₂ which was added after addition of DDW, as described above. Weight per weight percentages of all ingredients for the limonene-based terpenosome containing FITC in the absence and in the presence of MgCl₂ are listed in Table 24 below.

TABLE 24
Terpenosome (limonene) compositions of the invention
and control composition containing FITC

| | Ex. 129a | Ex. 129b | |
|---|---|---|---|
| | (of the | (of the | |
| | invention, | invention, | Ex. 129c |
| | not containing | containing | (compar- |
| | magnesium) | magnesium) | ative) |
| Ingredients | % w/w | % w/w | % w/w |
| FITC | 0.05 | 0.05 | 0.05 |
| Limonene | 5.00 | 5.00 | 5.00 |
| Glycyrrhizin | 2.00 | 2.00 | — |
| MgCl₂ | — | 0.20 | — |
| EtOH | 74.00 | 73.80 | — |
| Cetyl alcohol | — | — | 4.22 |
| Polysorbate 80 (Tween 80) | — | — | 12.67 |
| Sorbitan monostearate | — | — | 10.56 |
| (Span 60) | | | |
| Castor oil | — | — | 26.39 |
| DDW | 18.95 | 18.95 | 41.11 |

In addition, a control (non-vesicular) comparative composition containing FITC and limonene was prepared (Ex. 129c), as set out in Table 24 above. Briefly, sorbitan monostearate (also termed herein “Span 60”), cetyl alcohol and castor oil were mixed and melted together on top of a boiling water bath. In a separate vessel, Tween 80 was added to DDW and mixed well with vortex. The mixture was then heated in a 65 °C oven. The aqueous phase was added in a dropwise manner to the oily phase through mixing with Heidolph overhead stirrer at 500 RPM. The mixing was continued until the composition completely cooled (21 minutes). Limonene was then added and mixed well with a spatula. Two hours before starting the experiment, FITC was added and mixed well with the other components.

The ability of the compositions to enhance the permeation of FITC through the skin was tested in six Franz static diffusion cells using porcine skin, and the treated skin samples were scanned using a confocal laser scanning microscope (CLSM), as described above, using 1% laser intensity.

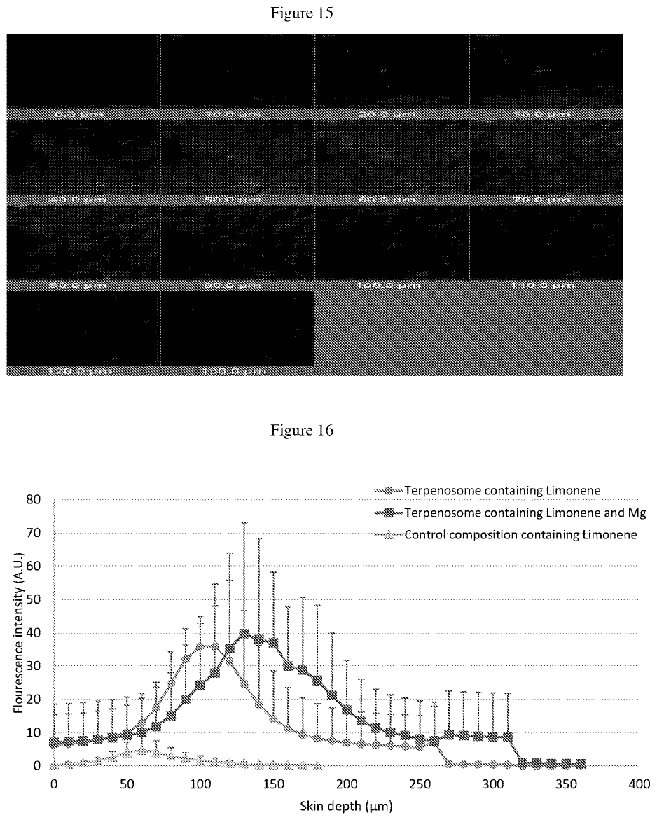

The results of the above experiments are shown in FIGS. 13, 14 and 15, presenting CLSM micrographs of skins treated with (1) terpenosome composition containing limonene (without addition of magnesium chloride); (2) terpenosome composition containing limonene and magnesium chloride; and (3) control composition containing limonene, respectively. The florescence values as a function of skin strata depth are presented in FIG. 16.

The results show that the terpenosome composition containing magnesium enhanced the penetration into deeper skin layers relative to terpenosome without magnesium and to the control treatment. The most intense fluorescence (39.7 A.U.) was seen at a skin depth 130 μm for the skin treated with terpenosome containing magnesium, while the terpenosome composition without magnesium chloride resulted in a maximum fluorescence intensity of 35.9 A.U. at 100 μm skin depth. The control treatment resulted in a maximum intensity of only 4.7 A.U. at a skin depth of 60 μm, with a zero intensity at skin depths of more than 100 μm.

It is clear that the presence of magnesium in the terpenosome composition allowed a deeper penetration of the probe into the skin.

### Example 130

### Effect of Magnesium on Skin Penetration of Molecules Incorporated in Terpenosomes—a CLSM Study

The effect of magnesium in a terpenosome composition on the skin penetration of FITC was also evaluated using Franz diffusion cell and CLSM analysis in a geraniol-based terpenosome. The lipophilic fluorescent molecule FITC (log p 4.3) was incorporated in a geraniol-based terpenosome, with and without magnesium, prepared as described above, as well as in a control composition, containing the same geraniol concentration.

First, geraniol-based terpenosome compositions containing FITC, in the absence of MgCl₂ (Ex. 130a) and in the presence of MgCl₂ (Ex. 130b), were prepared as described above and as set out in Table 25 below. FITC was added to the compositions and mixed well, as described above.

TABLE 25
Terpenosome (geraniol) compositions of the invention
and control composition containing FITC

| | Ex. 130a | Ex. 130b | |
|---|---|---|---|
| | (of the | (of the | |
| | invention, | invention, | Ex. 130c |
| | not containing | containing | (compar- |
| | magnesium) | magnesium) | ative) |
| Ingredients | % w/w | % w/w | % w/w |
| FITC | 0.05 | 0.05 | 0.05 |
| Cetyl alcohol | 0.5 | 0.5 | 4 |
| Geraniol | 10 | 10 | 10 |
| Glycyrrhizin | 2 | 2 | — |
| MgCl₂ | — | 0.2 | — |
| EtOH | 70 | 69.8 | — |
| Polysorbate 80 (Tween 80) | — | — | 12 |
| Sorbitan monostearate | — | — | 10 |
| (Span 60) | | | |
| Castor oil | — | — | 25 |
| DDW | 17.45 | 17.45 | 38.95 |

In addition, a non-vesicular composition was prepared, based on geraniol as set out in Table 25 above (Ex. 130c). Briefly, Span 60, cetyl alcohol and castor oil were mixed and melted together on top of boiling water bath. In a separate vessel, Tween 80 was added to DDW and mixed well with vortex. The mixture was then heated in a 65 °C oven. The aqueous phase was then added in a dropwise manner to the oily phase through mixing with Heidolph overhead stirrer at 500 rpm. The mixing was continued until the composition completely cooled (21 minutes). Geraniol was then added and mixed well, with a spatula. Two hours before starting the experiment, FITC was added and mixed well.

Next, the ability of the compositions to enhance the permeation of FITC through skin was tested in six Franz static diffusion cells, using porcine skin and the skin was monitored by CLSM, as described above (e.g., in Example 120), using 2% laser intensity.

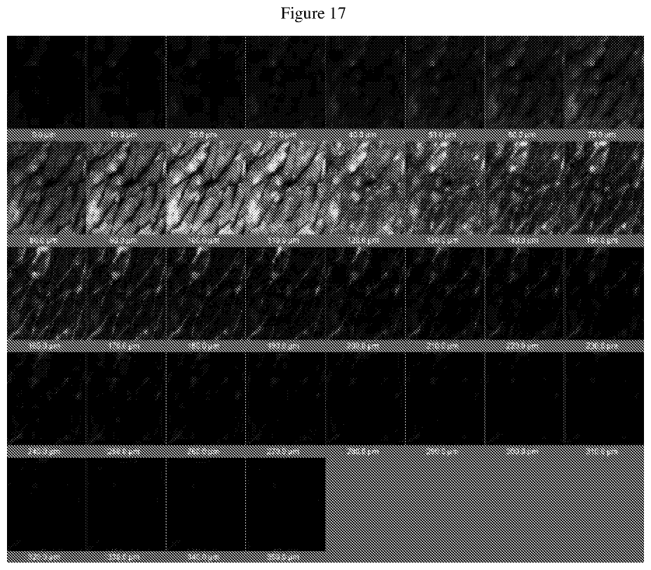

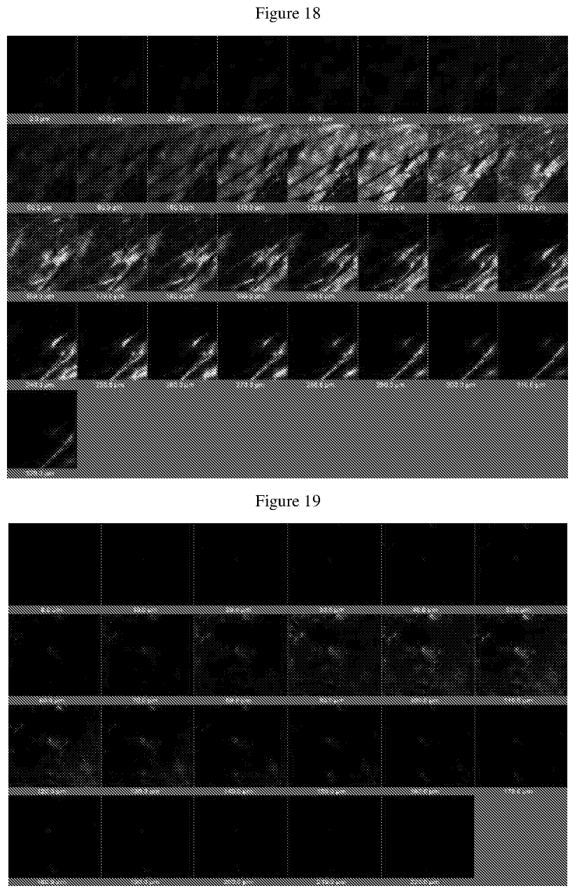

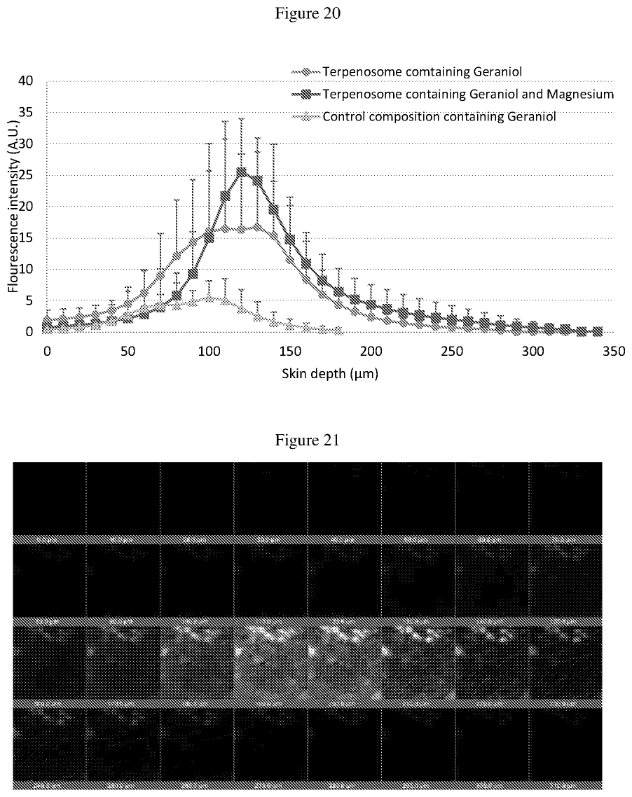

The results of the above experiments are shown in FIGS. 17, 18 and 19, presenting CLSM micrographs of skins treated with (1) terpenosome composition containing geraniol (without addition of magnesium chloride); (2) terpenosome composition containing geraniol and magnesium chloride; and (3) control composition containing geraniol, respectively. The florescence values as a function of skin strata depth are presented in FIG. 20.

The results show that the terpenosome composition containing magnesium enhanced the penetration into deeper skin layers relative to terpenosome without magnesium and to the control treatment. The maximum fluorescence intensity for the two terpenosome systems was measured at skin depth of 130 μm with superior values of 25.5 A.U for the system containing magnesium as compared to 16.7 A.U. for terpenosome composition without magnesium. The control non-vesicular composition resulted in a maximum fluorescent intensity of only 5.4 A.U. at a skin depth of 100 μm.

It is clear the presence of magnesium in the terpenosome composition allowed a deeper penetration of the probe into the skin.

### Example 131

### Comparative Skin Penetration of a Lipophilic Molecule Incorporated in Terpene Mixture-Based Terpenosomes—a CLSM Study

Next, the in vitro skin penetration of the lipophilic molecule FITC was evaluated when delivered from terpenosome systems listed in Table 26 below, containing either a terpene mixture or only one terpene (i.e., geraniol or limonene), at the same total concentration (of 5%). The experiment was carried out in Franz diffusion cells and tested by CLSM, as described above.

First, compositions were prepared as detailed above. Briefly, a single terpene or a terpene mixture was dissolved in ethanol using an overhead stirrer, at 500 RPM. The speed was lowered to 300 RPM, and glycyrrhizin was added through mixing, which was continued until the glycyrrhizin completely dissolved. DDW was then slowly and gradually added through mixing, which was continued for further five minutes. Two hours before starting the experiment, FITC was added to the above components and mixed well.

TABLE 26
Terpenosomes containing FITC

| Ex. 131a | Ex. 131b | Ex. 131c |
|---|---|---|
| Terpene | Geraniol | Limonene |
| mixture (5%) | (5%) | (5%) |

| | |
|---|---|
| Ingredients | % w/w |

| | | | |
|---|---|---|---|
| FITC | 0.05 | 0.05 | 0.05 |
| Limonene | 1.25 | — | 5 |
| Menthol | 1.25 | — | — |
| Geraniol | 1.25 | 5 | — |
| Eucalyptol | 1.25 | — | — |
| Glycyrrhizin | 2 | 2 | 2 |
| EtOH | 74 | 74 | 74 |
| DDW | 18.95 | 18.95 | 18.95 |

The ability of the compositions to enhance the permeation of FITC through the skin was tested in six Franz static diffusion cells using porcine skin, and skin was then monitored by CLSM, as described above, using 2% laser intensity.

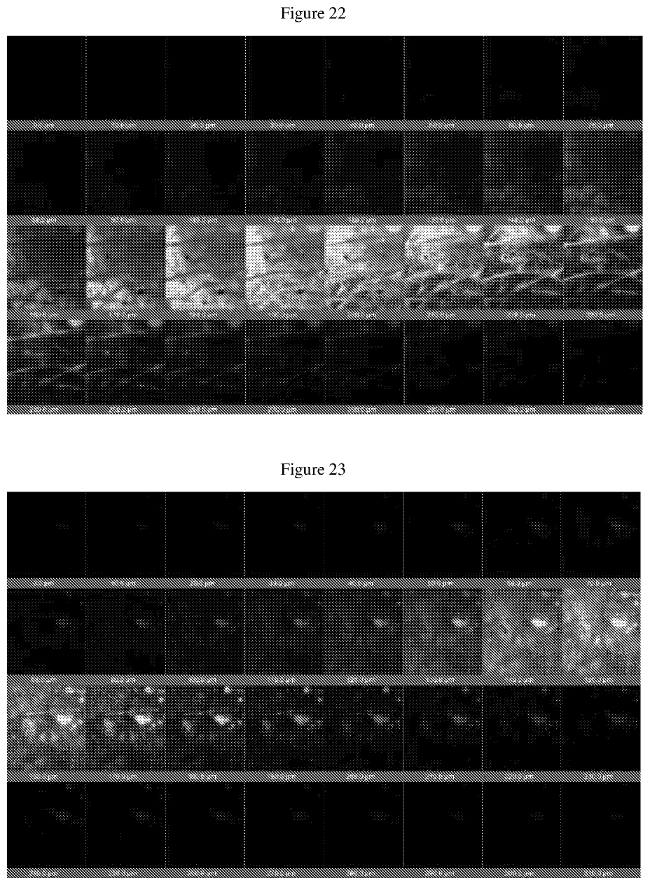

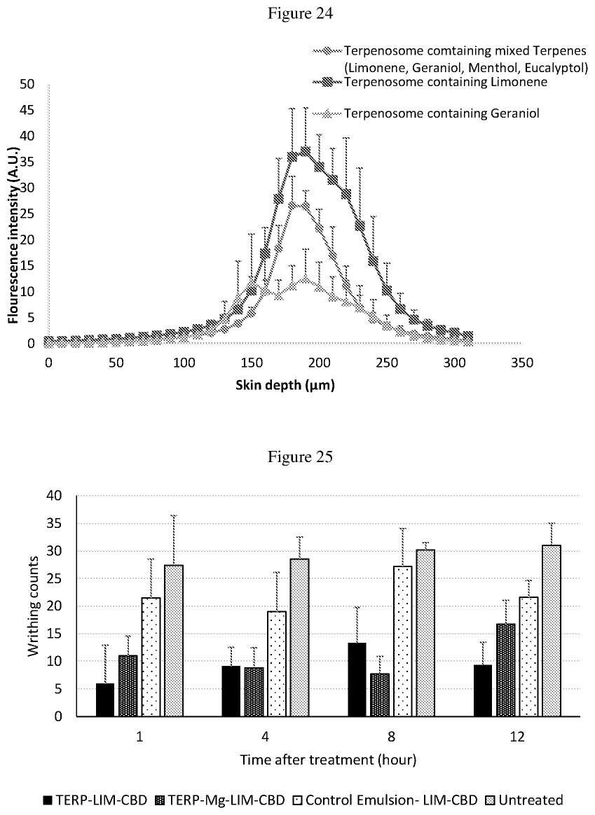

The results of the above experiments are shown in FIGS. 21-23, presenting CLS micrographs of skins treated with (1) terpenosome composition containing terpene mixture; (2) terpenosome composition containing limonene; and (3) terpenosome composition containing geraniol, respectively. The fluorescence values as a function of skin strata depth are plotted in FIG. 24.

The results show that all tested terpenosome enhanced the penetration of FITC into the deeper skin layers. The most intense fluorescence was observed at a skin depth of 150-220 microns for the three systems, with a maximum fluorescence intensity at 190 μm of 37.03, 28.46 and 12.58 A.U. for terpenosome compositions containing limonene, mixed terpenes and geraniol, respectively. The calculated total fluorescent intensity, expressed as AUC, was found to be 1767, 3222 and 1281 for terpenosome compositions containing mixed terpenes, limonene and geraniol, respectively.

### Example 132

Comparative Analgesic Effect of CBD Following Administration from Various Systems as a Function of Time—Induced Pain Animal Model

The analgesic effect of CBD administrated from various systems, 1, 4, 8 and 10 hours after administration was evaluated in an induced pain animal model, for the following composition (listed in Table 27 below):
1. Terpenosome containing 5% limonene and 5% CBD;
2. Terpenosome containing 5% limonene, magnesium chloride and 5% CBD; and
3. A control system (emulsion) containing 5% limonene and 5% CBD.

In addition, animals with induced pain and without analgesic treatment served as untreated control.

The compositions were prepared as described above. Briefly, terpenosome containing limonene and CBD in the absence (Ex. 132a) or in the presence of magnesium chloride (Ex. 132b) were prepared by first dissolving limonene in EtOH, then adding glycyrrhizin through mixing, which was continued until the glycyrrhizin completely dissolved. DDW was then slowly and gradually added through mixing which was continued for further five minutes. When present, MgCl₂ was then added and the mixing continued for additional five minutes. CBD was finally added and mixed until homogenous appearance.

TABLE 27
Terpenosome (limonene) compositions of the invention
and control composition containing CBD

| | Ex. 132a | Ex. 132b | |
|---|---|---|---|
| | (of the | (of the | Ex. 132c |
| | invention) | invention) | (comparative) |
| Ingredients | % w/w | % w/w | % w/w |
| CBD | 5.00 | 5.00 | 5 |
| Limonene | 4.750 | 4.750 | 5 |
| Glycyrrhizin | 1.900 | 1.900 | — |
| MgCl₂ | — | 0.190 | — |
| EtOH | 70.800 | 70.61 | — |
| Cetyl alcohol | — | — | 5 |
| Polysorbate 80 (Tween 80) | — | — | 5 |
| Sorbitan monostearate (Span 60) | — | — | 5 |
| Castor oil | — | — | 5 |
| DDW | 17.55 | 17.55 | 39 |

Next, control (comparative) emulsion containing limonene and CBD was prepared as generally described above and set out in Table 27 (Ex. 132c). Briefly, Span 60, Cetyl alcohol and castor oil were mixed and melted together on top of boiling water bath. In a separate vessel Tween 80 was added to DDW and mixed well with vortex. The mixture was then heated in a 65 °C oven. The aqueous phase was added in a dropwise manner to the oily phase through mixing with Heidolph overhead stirrer at 500 rpm. The mixing was continued until the composition completely cooled (21 min). CBD was mixed with limonene, and the mixture was added to the emulsion base gradually thorough mixing using a spatula.

The experiment was performed on 112 female CD-1 ICR mice (a mice model, as described in Allon, I. and Touitou, E., 2016. Scrolls: novel microparticulate systems for enhanced delivery to/across the skin. Drug delivery and translational research, 6(1), pp. 24-37). Mice were maintained under standard conditions, as detailed above and randomly and equally divided into four groups. One day before the experiment, 1.5*1.5 cm² of the dorsal skin area of animal was clipped. On the day of the experiment, the animals in the first three groups were anesthetized shortly with Isoflurane® and treated with 100 mg/kg CBD in various compositions: terpenosome containing 5% limonene and 5% CBD, terpenosome containing 5% limonene, 5% CBD and magnesium and a control emulsion containing 5% limonene and 5% CBD (n=7/group). The systems were applied topically and occlusively to the shaved area, as described above. The animals were anesthetized again and injected intraperitoneally with acetic acid (0.6% v/v) at a dose of (10 ml/kg) one, four, eight and twelve hours after administration with CBD. The fourth group (n=7) served as untreated control, as described above. Then, the number of writhing episodes was recorded and the analgesic effect of each treatment was expressed by the Maximum Possible Effect (MPE %) of the treatments, as described above.

The results of this in vivo evaluation of the analgesic effect of CBD administrated in terpenosome systems vs. control system are presented in Tables 28 and 29 below and in FIG. 25.

TABLE 28
Mean number of writhing episodes in mice groups topically treated
with 100 mg/kg CBD from terpenosomes containing limonene and CBD in the
absence or in the presence of MgCl₂ and from a control (“non-
terpenosome”) emulsion containing limonene and CBD, 1, 4, 8 and
12 hours prior to i.p. injection of acetic acid (Mean ± SD)

| | Time point (hours) | | | |
|---|---|---|---|---|
| | 1 | 4 | 8 | 10 |
| System | Writhes count | | | |
| Terpenosome containing limonene | 6.0 ± 6.9 | 9.2 ± 3.4 | 13.3 ± 6.5 | 9.3 ± 4.1 |
| and CBD (Ex. 132a) | | | | |
| Terpenosome containing | 11.0 ± 3.6 | 8.8 ± 3.7 | 7.7 ± 3.2 | 16.7 ± 4.3 |
| limonene, magnesium chloride | | | | |
| and CBD (Ex. 132b) | | | | |
| Comparative “non-terpenosome” | 21.5 ± 7.0 | 19.0 ± 7.1 | 27.2 ± 6.8 | 21.6 ± 3.0 |
| emulsion containing limonene and | | | | |
| CBD (Ex. 132c) | | | | |
| Untreated control | 27.4 ± 9.0 | 28.5 ± 4.0 | 30.2 ± 31.3 | 31 ± 4.0 |

TABLE 29
Mean MPE % values in mice groups topically treated with
100 mg/kg CBD from terpenosomes containing limonene
and CBD, in the absence or in the presence of MgCl₂,
or from a control (“non-terpenosome”) emulsion
containing limonene and CBD, 1, 4, 8 and 12 hours
prior to IP injection of acetic acid

| | Time point (hours) | | | |
|---|---|---|---|---|
| | 1 | 4 | 8 | 12 |
| System | MPE % | | | |
| Terpenosome containing limonene | 78.1 | 67.8 | 55.6 | 69.9 |
| and CBD (Ex. 132a) | | | | |
| Terpenosome containing | 59.9 | 69.0 | 76.3 | 45.2 |
| limonene, magnesium chloride | | | | |
| and CBD (Ex. 132b) | | | | |
| Control “non-terpenosome” | 21.5 | 33.3 | 9.9 | 30.3 |
| emulsion containing limonene and | | | | |
| CBD (Ex. 132c) | | | | |

As shown above, mice groups treated with terpenosome containing limonene and CBD, and with terpenosome containing limonene, CBD and magnesium chloride exhibited a significantly lower writhes count as compared to animals treated with the control emulsion (containing the same concentration of limonene and CBD) and as compared to the untreated animal group. This superior effect of CBD when administrated in terpenosome systems was already observed at the first testing time point (1 hour) where the writhes count was reduced from 27.4±9.0 for the untreated animals to 6.0±6.9 and 11.0±3.6 writhes for those received terpenosome containing limonene and CBD and terpenosome containing limonene, CBD and magnesium chloride, respectively. A weak analgesic effect was obtained at this time point following treatment with control emulsion containing limonene and CBD (27.4±9.0). This behavior of the systems was maintained throughout the 12 hours experiment. For example, eight hours after treatment, the MPE % values were 55.6, 76.3 and 9.9 for the terpenosome, terpenosome with magnesium chloride and control emulsion, respectively.

This data, schematically presented in FIG. 25, indicates the ability of the new terpene transdermal systems, terpenosomes, to enhance the analgesic effect of CBD. The weak effect demonstrated for the control emulsion containing the same concentration of limonene and CBD emphasizes the important role of the terpenosome system in drug delivery.

### Example 133

### Comparing the Analgesic Effect of Terpenosomes Containing Terpene Mixture and CBD to the Effect of a Control Emulsion—Induced Pain Animal Model

The analgesic effect of CBD administrated from various systems, one hour after administration, was next evaluated in the induced pain animal model for the following compositions (as set our in Table 30):
1. Terpenosome composition containing 5% terpene mixture and 5% CBD, applied occlusively;
2. Terpenosome composition containing 5% terpene mixture and 5% CBD, applied non-occlusively; and
3. Control (comparative) non-terpenosome system (emulsion) containing 5% terpene mixture and 5% CBD, applied non-occlusively.

In addition, animals induced with pain but lacking an analgesic treatment served as untreated control. Compositions were prepared as generally described above (Ex. 133a). Briefly, limonene, menthol, geraniol, eucalyptol and cetyl alcohol were dissolved in EtOH using an overhead stirrer at 500 RPM. The speed was lowered to 300 RPM, and glycyrrhizin was added through mixing, which was continued until the glycyrrhizin completely dissolved. DDW was then slowly and gradually added through mixing which was continued for further five minutes. CBD was then added and mixed well.

TABLE 30
Terpenosome composition containing mixed terpenes and CBD

| | Ex. 133a | Ex. 133b |
|---|---|---|
| | (of the invention) | (comparative) |
| Ingredients | % w/w | % w/w |
| CBD | 5 | 5 |
| Limonene | 1.25 | 1.25 |
| Menthol | 1.25 | 1.25 |
| Geraniol | 1.25 | 1.25 |
| Eucalyptol | 1.25 | 1.25 |
| Glycyrrhizin | 2 | — |
| Cetyl Alcohol | 0.5 | 4 |
| Polysorbate 80 (Tween 80) | — | 12 |
| Sorbitan monostearate (Span 60) | — | 10 |
| Castor oil | — | 25 |
| EtOH | 74 | — |
| DDW | 18.5 | 39 |

In addition, a comparative “non-terpenosome” control composition was prepared as described above and set out in Table 30 (Ex. 133b). Briefly, Span 60, cetyl alcohol and castor oil were mixed and melted together on top of boiling water bath. In a separate vessel, Tween 80 was added to DDW and mixed well with vortex. The mixture was then heated in a 65 °C oven. The aqueous phase was added in a dropwise manner to the oily phase through mixing with Heidolph overhead stirrer, at a speed of 500 RPM. Mixing was continued until the composition completely cooled. CBD, mixed with the terpenes, were then added to the emulsion and mixed well.

The experiment was performed on 24 female CD-1 ICR mice (Allon, I. and Touitou, E., supra). Mice were maintained under standard conditions, as detailed above. The animals were randomly and equally divided into four (4) groups. One day before the experiment, 1.5*1.5 cm² of the dorsal skin area of animal was clipped. On the day of the experiment, the animals in the first three groups were anesthetized shortly with Isoflurane® and treated with 100 mg/kg CBD from terpenosome containing 5% limonene and 5% CBD (applied occlusively to group 1 and non-occlusively to group 2) and from a control emulsion containing 5% limonene and 5% CBD (n=6/group). The animals were anesthetized again and injected intraperitoneally with acetic acid (0.6% v/v) at a dose of (10 ml/kg) one hour after treatments. The fourth group (n=7) served as untreated control. Animals in this group were anesthetized with Isoflurane® injected with acetic acid at the same dose without drug treatment. The number of writhing episodes was recorded and the analgesic effect of each treatment was expressed by the Maximum Possible Effect (MPE %) of the treatments, as detailed above.

The results of this in vivo evaluation of the analgesic effect of CBD administrated in terpenosome composition containing a terpene mixture vs. a non-terpenosome control formulation are presented in Table 31 and in FIG. 26.

TABLE 31
Mean MPE % values in mice pain model groups treated with 100 mg/kg
CBD topically from terpenosome containing 5% mixed terpenes and
CBD (occluded or non- occluded) or from control non-terpenosome
emulsion containing 5% mixed terpenes (Non-occluded).

| Treatment | MPE % |
|---|---|
| Terpenosome containing 5% mixed terpenes + | 72.9 |
| CBD (Ex. 133a, Occluded) | |
| Terpenosome containing 5% mixed terpenes + | 72.9 |
| CBD (Ex. 133a, Non-occluded) | |
| Control a non- terpenosome emulsion containing 5% | 21.3 |
| mixed terpenes (Ex. 133b, Non-occluded) | |

The results indicate that topical administration of CBD in terpenosome systems (occluded or non-occluded) one hour prior to the pain induction improved significantly the analgesic effect, with MPE values of 72.9%. These values are in comparison with emulsion containing the same terpene mixture at an equal concentration, which yielded an MPE of only 21.3%. No difference was observed between the occluded and the non-occluded application.

### Example 134

### Effect of Cetyl Alcohol on Skin Penetration of Molecules Incorporated in Terpenosomes

Next, the effect of the presence of cetyl alcohol in terpenosome on the skin penetration of incorporated molecules was evaluated, by following the skin penetration of FITC incorporated into terpenosome compositions prepared in the absence and in the presence of cetyl alcohol as set out in Table 32 below.

TABLE 32
Terpenosome Composition comprising FITC
prepared with or without Cetyl alcohol

| | Ex. 134a | Ex. 134b |
|---|---|---|
| Ingredients | % w/w | % w/w |
| FITC | 0.05 | 0.05 |
| Cetyl alcohol | 0.5 | — |
| Geraniol | 5 | 5 |
| Glycyrrhizin | 2 | 2 |
| EtOH | 74 | 74.4 |
| DDW | 18.45 | 18.55 |

The compositions were prepared as described above. Briefly, geraniol was dissolved in EtOH using an overhead stirrer and mixed at 700 RPM. Glycyrrhizin was added through mixing, which was continued until it completely dissolved. DDW was then slowly and gradually added through mixing which was continued for further five minutes and FITC was added and mixed well two hours before starting the experiment. When present, cetyl alcohol was first mixed with geraniol and then both were dissolved in EtOH and the preparation was continued as above.

The above compositions were next evaluated in an in vitro skin penetration assay carried out in Franz diffusion cells, and the results were analyzed using confocal laser scanning microscopy (CLSM), as described above. Briefly, skin pieces were placed on the cells donor and left to warm for 15 minutes. The receiver was filled with 30% hydroethanolic solution. Composition (25 μl of each) were applied on the epidermal side of the skin under non-occlusive conditions for one hour, in a dark room. Blank skin sample served as control, to rule out auto-fluorescence of the skin tissue. Then, the skin was removed from the cells and its surface was carefully washed twice with DDW (600 μl) and Kimwipes (Kimberly-Clark, Canada). The permeation area was then cut and mounted between two cover slips of 24×60 mm for microscopic examination.

Samples were then scanned using a confocal laser scanning microscope (Zeiss LSM 980 laser scanning microscopy system, Zeiss, Germany) at a stack scanning mode, 2% laser intensity, 10 m increments through the z-axis with an air plane ×10 objective lens and 488 nm excitation wavelength. During the microscopic examination, each skin sample was divided to 5×5 tiles and micrographic images were obtained. The fluorescence intensity (arbitrary units) was assessed using the ImageJ software.

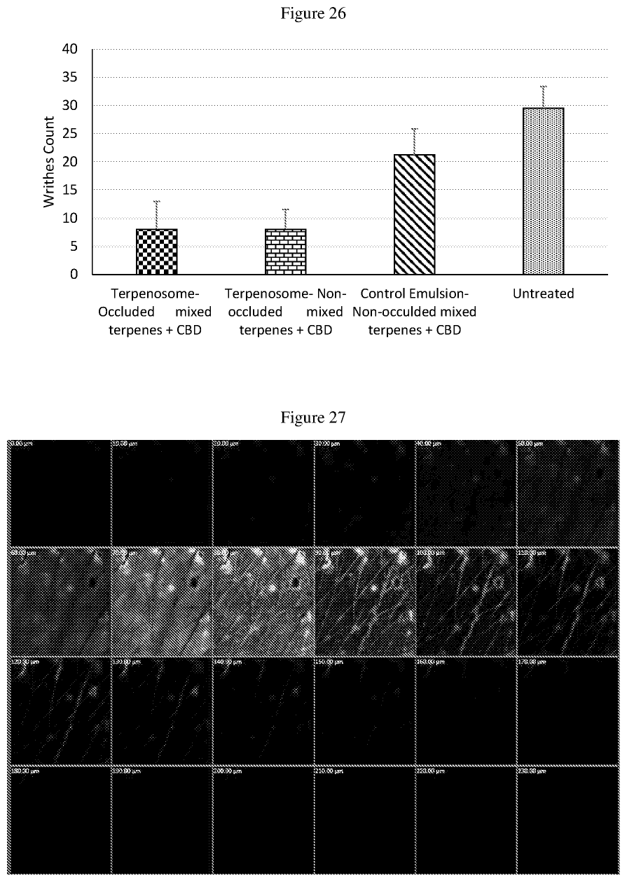

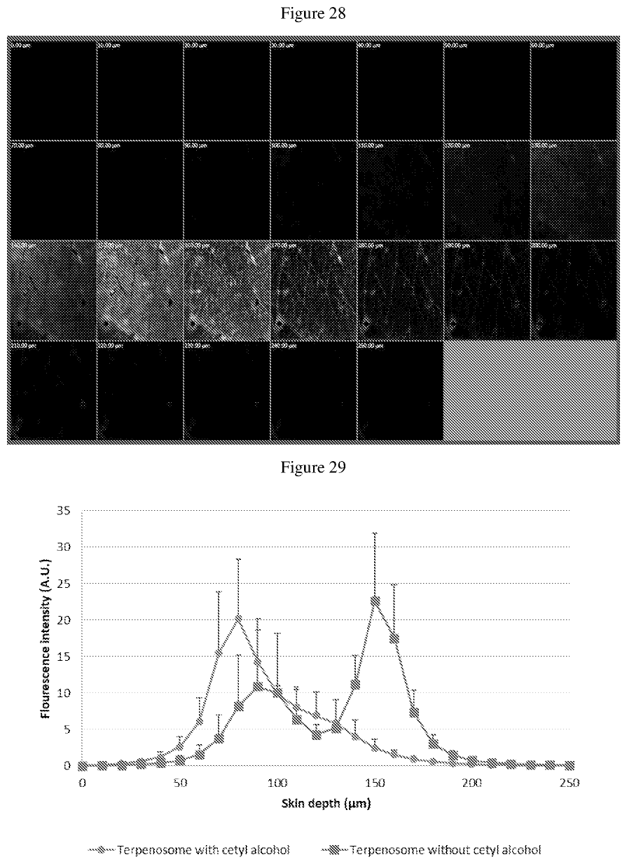

FIG. 27 presents CLS micrographs of skin treated with a terpenosome composition containing cetyl alcohol and FIG. 28 presents CLS micrographs of skin treated with a terpenosome composition without cetyl alcohol. The florescence values as a function of skin strata depth are plotted in FIG. 29.

The results indicate that the composition without cetyl alcohol delivered the incorporated molecule deeper into the skin layers (i.e., trans-dermal delivery), whereas the composition containing cetyl alcohol delivered the incorporated molecule into upper skin layers, namely skin layers closer to the skin upper surface (i.e., dermal delivery). Terpenosome composition containing cetyl alcohol delivered the probe showing a maximum fluorescent intensity of 20.2 A.U. at a skin depth of 80 μm. On the other hand, the terpenosome without cetyl alcohol delivered the probe deeper into the skin with a maximum fluorescence intensity of 22.6 at a skin depth of 150 μm. It is noteworthy that the area under the curve (AUC) values for both formulations were similar, 1166.7 and 1020.6 A.U.*μm, respectively.

Based on the results presented above, terpenosomes may be formulated for dermal or transdermal administration (or delivery) of active molecules.

### Example 135

### Visualization of the Vesicular Structure of Terpenosome by Transmission Electron Microscopy (TEM): Effect of the Presence of Cetyl Alcohol in the Composition

The effect of cetyl alcohol on the microscopic structure of terpenosomes was further studied on geraniol-based terpenosome formulations prepared as set out in Table 33 below.

TABLE 33
Terpenosome Composition without cetyl alcohol

| | Ex. 135a | Ex. 135b |
|---|---|---|
| Ingredients | % w/w | % w/w |
| Cetyl alcohol | — | 0.5 |
| Geraniol | 5 | 5 |
| Glycyrrhizin | 2 | 2 |
| EtOH | 74.4 | 74 |
| DDW | 18.6 | 18.56 |

The compositions were prepared as described above, briefly, geraniol was dissolved in EtOH in a closed glass vessel using an overhead stirrer and mixed at 700 RPM. Glycyrrhizin was then added through mixing, which was continued until it completely dissolved. DDW was then slowly and gradually added through mixing which was continued for further 5 minutes. When present, cetyl alcohol was first mixed with geraniol and then both were dissolved in EtOH and preparation was continued as above.

Prior to the transmission electron microscopic examination, the geraniol-based terpenosome systems were 1:10 diluted with EtOH:DDW (4:1) solution. Samples (3 l) were then placed on a 400 mesh carbon coated grid and stained with 1% aqueous solution of phosphotungstic acid (PTA) for 20 seconds, dried for 20 minutes at room temperature, then visualized using Philips TECHNAI CM 120 (Eindhoven, Netherland).

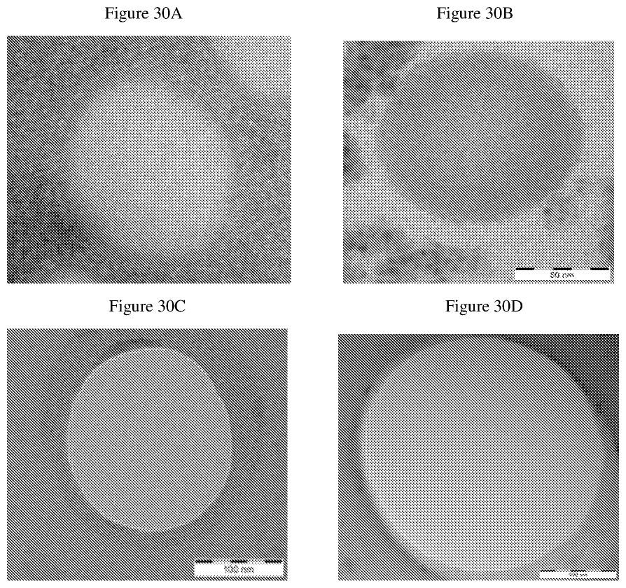

As shown in FIG. 30A through FIG. 30D, terpenosomes prepared with (FIG. 30C and FIG. 30D) or without (FIG. 30A and FIG. 30B) cetyl alcohol formed spherical, uni-lamellar nano-vesicles. The results of this experiment indicate that the presence of cetyl alcohol resulted in a more evident, possibly stronger, lamella, as shown in FIG. 30C and FIG. 30D.

1. A composition comprising at least one active agent in a vesicular carrier, wherein the vesicular carrier comprises at least one non-saponin terpene, at least one saponin, at least one C₂-C₄ alcohol and water, wherein said composition is substantially phospholipid-free.

2. The vesicular composition according to claim 1, for dermal or transdermal delivery.

3. The vesicular composition according to claim 1, wherein the active agent is selected from analgesic agents and/or agents for treating and/or preventing at least one skin-related disease or condition.

4. The vesicular composition according to claim 1, wherein the active agent is a cannabinoid.

5. The vesicular composition according to claim 1, wherein the composition further comprises a long chain aliphatic alcohol.

6. The vesicular composition according to claim 5, wherein the long chain aliphatic alcohol is cetyl alcohol.

7. The vesicular composition according to claim 1, wherein the composition further comprises magnesium salt.

8. The vesicular composition according to claim 7, wherein the magnesium salt is magnesium chloride.

9. The vesicular composition according to claim 1, wherein the C₂-C₄ alcohol is ethanol.

10. The vesicular composition according to claim 1, wherein the terpene is selected from monoterpenes and sesquiterpenes.

11. The vesicular composition according to claim 10, wherein the monoterpene is acyclic monoterpene.

12. The vesicular composition according to claim 11, wherein the acyclic monoterpene contains one or more oxygen atoms.

13. The vesicular composition according to claim 12, wherein the acyclic monoterpene is selected from the group consisting of geraniol, citronellol and linalool.

14. The vesicular composition according to claim 10, wherein the monoterpene is cyclic monoterpene.

15. The vesicular composition according to claim 14, wherein the cyclic monoterpene is devoid of oxygen atoms.

16. The vesicular composition according to claim 15, wherein the cyclic monoterpene devoid of oxygen atoms is limonene.

17. The vesicular composition according to claim 14, wherein the cyclic monoterpene contains one or more oxygen atoms.

18. The vesicular composition according to claim 17, wherein the cyclic monoterpene contains one or more ring oxygen heteroatom(s).

19. The vesicular composition according to claim 18, wherein the cyclic monoterpene is eucalyptol.

20. The vesicular composition according to claim 14, wherein the cyclic monoterpene contains one or more hydroxyl groups attached to aliphatic ring carbon.

21. The vesicular composition according to claim 20, wherein the cyclic monoterpene is menthol.

22. The vesicular composition according to claim 1, wherein the saponin is glycyrrhizin, a ginsenoside or a Quillaja saponin.

23. (canceled)

24. The vesicular composition according to claim 1, further comprising at least one polyol.

25. (canceled)

26. The vesicular composition according to claim 1, comprising:
from about 0.005% to about 10% by weight of at least one active agent selected from analgesics, agents for treating and/or preventing at least one skin-related disease or condition and melatonin;
from about 0.1% to about 20% by weight of at least one terpene selected from monoterpenes and sesquiterpenes;
from about 0.1% to about 5% by weight of at least one saponin;
from about 15% to about 80% by weight of ethanol;
when present, from about 0.05% to about 5% by weight of long chain alcohol;
when present, up to 60% by weight propylene glycol and/or 1,3-propanediol;
when present, from about 0.01% to about 0.5% by weight of magnesium salt;
and
from about 5% to about 85% by weight of water.

27. (canceled)

28. The vesicular composition according to claim 26, wherein said agents for treating and/or preventing at least one skin-related disease or condition are selected from anti-Acne agents, anti-aging agents, skin exfoliating agents and anti-wrinkles agents.

29. (canceled)

30. A method for treatment and/or prevention of a disease or condition in a subject comprising administering to the skin of said subject a therapeutically effective and/or prophylactically effective amount of a composition according to claim 1.

31. The method according to claim 30, wherein said condition is pain and wherein the active agent is an analgesic.

32. (canceled)

33. The method according to claim 30, wherein said disease or condition is associated with upper skin layers.

34. The method according to claim 33, wherein said disease or condition is Acne, rosacea, skin infection, Contact dermatitis, Atopic dermatitis, Actinic keratosis, skin inflammation, Psoriasis, Desquamation, Exfoliation, a cosmetic condition, damaged skin barrier, or dehydrated skin.

35. The method according to claim 33, wherein the composition administered to the subject comprises a long chain aliphatic alcohol.

36. The method according to claim 30, wherein said disease or condition is associated with deeper skin layers.

37. The method according to claim 36, wherein said disease or condition is Acne, pain, Alopecia Areata, Raynaud's Phenomenon, Vitiligo, Scleroderma, Hidradenitis suppurativa (acne inversa), Melanoma, Squamous cell carcinoma, Basal cell carcinoma, Cellulitis, Pemphigus, Psoriasis, skin regeneration, Collagen activation, skin pigmentation or skin hyperpigmentation.

38. The method according to claim 36, wherein the composition administered to the subject comprises magnesium salt.

39. A method of cosmetic treating and/or prevention of at least one skin condition in a subject, comprising applying to the skin of said subject a composition according to claim 1.

40. (canceled)

41. (canceled)

42. A phospholipid-free vesicular composition comprising at least one non-saponin terpene, at least one saponin, at least one C₂-C₄ alcohol and water, wherein the composition further comprises a long chain alcohol and/or a magnesium salt, wherein the composition is devoid of non-terpene active agent.
